SEQUENCE LISTING (8 sequences)

SEQ ID NO: 1 - skipped

SEQ ID NO: 2 - protein, 9 aa, synthetic construct
GGIGGIGGI

SEQ ID NO: 3 - DNA, 16 bp, synthetic construct
  source 1..16: mol_type = other DNA
aaaaaaaaaaaaaaaa

SEQ ID NO: 4 - DNA, 12 bp, synthetic construct
  source 1..12: mol_type = other DNA
cccccccccccc

SEQ ID NO: 5 - DNA, 17 bp, synthetic construct
  source 1..17: mol_type = other DNA
ccccccccccccccccc

SEQ ID NO: 6 - DNA, 11 bp, synthetic construct
  source 1..11: mol_type = other DNA
aaaaaaaaaaa

SEQ ID NO: 7-8 - skipped (2 entries)

(19) United States
(12) Patent Application Publication
(10) Pub. No.: US 2026/0022419 A1
(43) Pub. Date: Jan. 22, 2026

(54) COMPOSITIONS AND METHODS OF MAKING GENE EXPRESSION LIBRARIES

(71) Applicant: 10x Genomics, Inc., Pleasanton, CA (US)

(72) Inventors: Zachary Bent, Pleasanton, CA (US); Marlon Stoeckius, Stockholm (SE)

(73) Assignee: 10x Genomics, Inc., Pleasanton, CA (US)

(21) Appl. No.: 19/326,091

(22) Filed: Sep. 11, 2025

Related U.S. Application Data
(63) Continuation of application No. 18/490,390, filed on Oct. 19, 2023.
Application No. 18/490,390 is a division of application No. 17/162,991, filed on Jan. 29, 2021, now Pat. No. 11,821,035.
(60) Provisional application No. 62/967,361, filed on Jan. 29, 2020.
(60) Provisional application No. 63/033,566, filed on Jun. 2, 2020.

(51) Int. Cl. C12Q 1/6876

(52) U.S. Cl. CPC C12Q 1/6876

(57) ABSTRACT

Provided herein are methods of detecting target nucleic acids and uses of the same.

### CROSS-REFERENCE TO RELATED APPLICATIONS

This application is a continuation application of U.S. patent application Ser. No. 18/490,390, filed on Oct. 19, 2023, which is a divisional application of U.S. patent application Ser. No. 17/162,991, now U.S. Pat. No. 11,821,035, filed Jan. 29, 2021, which claims priority to U.S. Provisional Patent Application No. 62/967,361, filed Jan. 29, 2020, and U.S. Provisional Patent Application No. 63/033,566, filed Jun. 2, 2020; each of which is incorporated herein by reference in its entirety.

### SEQUENCE LISTING

This application contains a Sequence Listing that has been submitted electronically as an XML file named “47706-0154003_SL_ST26.XML.” The XML file, created on Sep. 11, 2025, is 5,978 bytes in size. The material in the XML file is hereby incorporated by reference in its entirety.

### BACKGROUND

Cells within a tissue of a subject have differences in cell morphology and/or function due to varied analyte levels (e.g., gene and/or protein expression) within the different cells. The specific position of a cell within a tissue (e.g., the cell's position relative to neighboring cells or the cell's position relative to the tissue microenvironment) can affect, e.g., the cell's morphology, differentiation, fate, viability, proliferation, behavior, and signaling and cross-talk with other cells in the tissue.

Spatial heterogeneity has been previously studied using techniques that only provide data for a small handful of analytes in the context of an intact tissue or a portion of a tissue, or provide a lot of analyte data for single cells, but fail to provide information regarding the position of the single cell in a parent biological sample (e.g., tissue sample).

### SUMMARY

This application is based on the discoveries of a method of making a spatial 5′ gene expression library and arrays for use in methods that allow for spatial analysis of large target analytes, e.g., V(D)J (or VDJ) rearranged T-cell receptors or immunoglobulins, a method for detecting and/or analyzing the 5′ sequence of a polynucleotide of interest, and a method for removing sequences from a polynucleotide of interest to facilitate detection and/or analysis of a 5′ sequence of the polynucleotide.

In one aspect, methods are provided for determining a location of a target nucleic acid in a biological sample that include: (a) contacting the biological sample with an array comprising a feature, wherein the feature comprises an attached first and second probe, wherein: a 5′ end of the first probe is attached to the feature; the first probe comprises in a 5′ to a 3′ direction: a spatial barcode and a capture domain (e.g., a poly(T) capture domain), wherein the poly(T) capture domain binds specifically to the target nucleic acid; a 5′ end of the second probe is attached to the feature; a 3′ end of the second probe is reversibly blocked; and the second probe comprises a poly(GI) capture domain; (b) extending a 3′ end of the first probe to add a sequence that is complementary to a portion of the target nucleic acid; (c) ligating an adapter to the 5′ end of the target nucleic acid specifically bound to the first probe; (d) adding a sequence complementary to the adapter to the 3′ end of the first probe; (e) adding non-templated cytosines to the 3′ end of the first probe to generate a poly(C) sequence, wherein the poly(C) sequence specifically binds to the poly(GI) capture domain of the second probe; (f) unblocking the 3′ end of the second probe and extending the 3′ end of the second probe to add a sequence comprising a sequence in the target nucleic acid and a sequence that is complementary to the spatial barcode; (g) cleaving a region of the second probe at a cleavage site that is 5′ to the poly(GI) capture domain, thereby releasing the second probe from the feature; and (h) determining (i) all or a part of the sequence of the spatial barcode, or a complement thereof, and (ii) all or a part of the sequence of the target nucleic acid, or a complement thereof, and using the sequences of (i) and (ii) to determine the location of the target nucleic acid in the biological sample.

In some embodiments of any of the methods described herein, the poly(GI) capture domain comprises a sequence of (GGI)n, wherein n is about 3 to about 20.

In some embodiments of any of the methods described herein, step (h) comprises sequencing all or a part of the sequence of the spatial barcode, or a complement thereof, and sequencing all of a part of the sequence of the target nucleic acid, or a complement thereof. In some embodiments of any of the methods described herein, the sequencing is performed using high throughput sequencing.

In some embodiments of any of the methods described herein, the first probe further comprises a unique molecular identifier (UMI), wherein the UMI in the first probe is positioned 5′ to the poly(T) capture domain in the first probe.

In some embodiments of any of the methods described herein, the second probe further comprises a unique molecule identifier (UMI), wherein the UMI in the second probe is positioned 5′ to the poly(GI) capture sequence in the second probe, wherein the UMI in the second probe is 3′ positioned relative to the cleavage site in the second probe.

In some embodiments of any of the methods described herein, the UMI in the first probe and the UMI in the second probe comprise different sequences. In some embodiments of any of the methods described herein, the UMI in the first probe and the UMI in the second probe comprise the same sequence.

In some embodiments of any of the methods described herein, the second probe further comprises an spatial barcode, wherein the spatial barcode in the second probe is 3′ positioned relative to the cleavage site in the second probe.

In some embodiments of any of the methods described herein, the spatial barcode in the first probe and the spatial barcode in the second probe comprise different sequences. In some embodiments of any of the methods described herein, the spatial barcode in the first probe and the spatial barcode in the second probe comprise the same sequence.

In some embodiments of any of the methods described herein, the target nucleic acid is RNA. In some embodiments of any of the methods described herein, the RNA is an mRNA. In some embodiments of any of the methods described herein, mRNA encodes a T-cell receptor.

Some embodiments of any of the methods described herein further include determining a sequence encoding one or more complementarity determining region(s) (CDR(s)) of the T-cell receptor.

In some embodiments of any of the methods described herein, the mRNA encodes an immunoglobulin heavy or light chain. In some embodiments of any of the methods described herein, the method further comprises determining a sequence encoding one or more CDR(s) of the immunoglobulin heavy or light chain.

In some embodiments of any of the methods described herein, the array is a bead array. In some embodiments of any of the methods described herein, the array is a slide.

In some embodiments of any of the methods described herein, the biological sample is a tissue section. In some embodiments of any of the methods described herein, the tissue section is a formalin-fixed and paraffin-embedded (FFPE) tissue section. In some embodiments of any of the methods described herein, the tissue section is a fresh frozen tissue section.

Also provided herein are arrays comprising a feature, where the feature comprises an attached first and second probe, wherein: a 5′ end of the first probe is attached to the feature; the first probe comprises in a 5′ to a 3′ direction: a spatial barcode and a poly(T) capture domain, where the poly(T) capture domain binds specifically to the target nucleic acid; a 5′ end of the second probe is attached to the feature; a 3′ end of the second probe is reversibly blocked; and the second probe comprises a poly(GI) capture domain. In some embodiments of any of the arrays described herein, the poly(GI) capture domain comprises a sequence of (GGI)n, wherein n is about 3 to about 20.

In some embodiments of any of the arrays described herein, the first probe further comprises a unique molecular identifier (UMI), wherein the UMI in the first probe is positioned 5′ to the poly(T) capture domain in the first probe.

In some embodiments of any of the arrays described herein, the second probe further comprises a unique molecule identifier (UMI), wherein the UMI in the second probe is positioned 5′ to the poly(GI) capture sequence in the second probe, wherein the UMI in the second probe is 3′ positioned relative to the cleavage site in the second probe.

In some embodiments of any of the arrays described herein, the UMI in the first probe and the UMI in the second probe comprise different sequences. In some embodiments of any of the arrays described herein, the UMI in the first probe and the UMI in the second probe comprise the same sequence.

In some embodiments of any of the arrays described herein, the second probe further comprises a spatial barcode, wherein the spatial barcode in the second probe is 3′ positioned relative to the cleavage site in the second probe.

In some embodiments of any of the arrays described herein, the spatial barcode in the first probe and the spatial barcode in the second probe comprise different sequences. In some embodiments of any of the arrays described herein, the spatial barcode in the first probe and the spatial barcode in the second probe comprise the same sequence.

In some embodiments of any of the arrays described herein, the array is a bead array. In some embodiments of any of the arrays described herein, the array is a slide.

In another aspect, a method is provided for detecting and/or determining a location of a biological analyte within a biological sample. The method comprises: (a) contacting a biological sample with a substrate, wherein the substrate comprises an attached first and second probe, wherein: a 5′ end of the first probe is attached to the substrate; the first probe comprises, in a 5′ to 3′ direction: a spatial barcode and a first capture domain; a 5′ end of the second probe is attached to the substrate; and the second probe comprises a second capture domain, wherein a target polynucleotide sequence of a biological analyte within the biological sample binds to the first capture domain; (b) extending a 3′ end of the first probe to generate an extension product that comprises a nucleotide sequence that is complementary to the target polynucleotide sequence, or a portion thereof; (c) attaching an adapter to the 3′ end of the extension product, wherein the adapter comprises a sequence that is complementary to the sequence of the second capture domain; (d) hybridizing the adapter to the second capture domain; and (e) extending a 3′ end of the second capture domain, thereby generating a nucleotide sequence that comprises a 5′ sequence of the target polynucleotide sequence of the biological analyte proximal to the sequence of the second capture domain.

In some embodiments, attaching the adaptor in step (c) comprises adding untemplated nucleotides to the 3′ end of the extension product, hybridizing a template switching oligonucleotide to the untemplated nucleotides, and extending the 3′ end of the extension product, thereby generating the adapter, wherein the adapter comprises a sequence that is complementary to the sequence of the template switching oligonucleotide. The untemplated nucleotides may include any nucleotides or nucleotide sequences thereof. In some embodiments, the untemplated nucleotides comprise a poly(C) or a poly(G) sequence.

In some embodiments, attaching the adaptor in step (c) includes ligating the adapter to the 3′ end of the extension product.

In some embodiments, step (b) and/or (e) comprises extending the 3′ end of the first probe or the second probe, respectively, with a reverse transcriptase or polymerase (e.g., RNA polymerase or DNA polymerase).

In some embodiments, step (a) further comprises permeabilizing the biological sample prior to or after contacting the substrate.

In various embodiments, the biological sample may be a tissue section, a primary cell, a cell line, or an organoid. For example, the biological sample may be a formalin-fixed and paraffin-embedded (FFPE) or paraformaldehyde (PFA) tissue section or a fresh frozen tissue section.

The target polynucleotide may comprise DNA, RNA. The first capture domain may comprise a poly(dT) sequence, a random sequence, or a sequence that is complementary to a target sequence of interest. In one embodiment, the target polynucleotide comprises mRNA. For example, the mRNA may encode a T-cell receptor or B-cell receptor, and in one embodiment, the method further comprises determining a sequence that encodes one or more complementarity determining region(s) (CDR(s)) of the T-cell receptor or B-cell receptor. For example, the mRNA may encode an immunoglobulin heavy or light chain, and in one embodiment, the method further comprises determining a sequence that encodes one or more CDR(s) of the immunoglobulin heavy or light chain.

In some embodiments, at least one of the first probe and the second probe further comprises a unique molecular identifier (UMI) positioned 5′ to the respective capture domain. In one embodiment, the first and second probes each comprise a UMI, and the UMI in the first probe and the UMI in the second probe comprise different sequences.

In some embodiments, the second probe further comprises a spatial barcode positioned 5′ to the second capture domain. In one embodiment, the spatial barcode in the first probe and the spatial barcode in the second probe comprise different sequences. In another embodiment, the spatial barcode in the first probe and the spatial barcode in the second probe comprise the same sequence.

In some embodiments, the substrate comprises an array comprising a feature, wherein the first and second polynucleotide probes are attached to the feature. For example, the array may be a bead array (for example, gel beads) or a slide.

In some embodiments, a plurality of first and second probes are attached to the substrate, and the ratio of first probes to second probes on the substrate is about 1:1000 to about 1000:1, or about 1:1 to about 1:100.

In some embodiment, the first and second probes are directly attached to the substrate. In other embodiments, the first and second probes are indirectly attached to the substrate. For example, the first and second probes may be attached to beads and the beads attached to the substrate.

The method may further comprise: (f) determining (i) all or a portion of the target polynucleotide sequence or a complement thereof, and (ii) the sequence of all or a portion of the spatial barcode, or a complement thereof, and using the determined sequences of (i) and (ii) to identify the location of the biological analyte in the biological sample. Prior to step (f), the method may comprise: generating an amplification product from the extension product generated in step (e), wherein the amplification product comprises the 5′ sequence of the target polynucleotide or the complement thereof, and wherein step (f) comprises determining the sequences of (i) and (ii) using the amplification product. Prior to step (f), the method may comprise: cleaving a region of the second probe at a cleavage site that is 5′ to the second capture domain, thereby releasing the second probe from the substrate.

In another aspect, a method is provided for detecting a biological analyte within a biological sample. The method comprises: (a) hybridizing first and second oligonucleotides to a template polynucleotide, wherein the template polynucleotide comprises a polynucleotide sequence of a biological analyte or a complement thereof; wherein: the first oligonucleotide comprises a sequence that is complementary to a first sequence of the template polynucleotide; the second oligonucleotide is a bridging oligonucleotide that comprises: sequences that are complementary to second and third sequences of the template polynucleotide, and a 5′ phosphate group, wherein the second and third sequences of the template polynucleotide are 3′ and 5′, respectively, to a fourth sequence that comprises a polynucleotide region to be removed from the template polynucleotide; wherein the first sequence of the template polynucleotide is 3′ to the second sequence of the template polynucleotide; (b) extending 3′ ends of the first and second oligonucleotides, thereby producing extended first and second oligonucleotides, wherein extension of the first oligonucleotide ceases when the 5′ phosphate of the second oligonucleotide is reached, thereby resulting in a nick between the extended first and second oligonucleotides; and (c) ligating the 3′ end of the extended first oligonucleotide and the 5′ phosphate of the extended second oligonucleotide, thereby producing a polynucleotide product that comprises a complement of 5′ and 3′ portions of the template polynucleotide sequence and does not comprise the complement of the fourth sequence of the template polynucleotide.

In some embodiments, the first sequence of the template polynucleotide to which the first oligonucleotide hybridizes is at the 3′ end of the template polynucleotide.

In some embodiments, the first sequence of the template polynucleotide to which the first oligonucleotide hybridizes is an adapter that is attached to the 3′ end of the template polynucleotide. In one embodiment, prior to step (a): (i) untemplated nucleotides are added to the 3′ end of the template polynucleotide; a template switching oligonucleotide is hybridized to the untemplated nucleotides; and (ii) the 3′ end of the template polynucleotide is extended, thereby generating the adapter, wherein the adapter is complementary to the sequence of the template switching oligonucleotide. The untemplated nucleotides may comprise any nucleotides or sequence of nucleotides. In some embodiments, the untemplated nucleotides comprise a poly(C) or poly(G) sequence. In another embodiment, prior to step (a), the adapter is ligated to the 3′ end of the template polynucleotide.

In some embodiments, step (b) comprises extending the 3′ ends of the first and second oligonucleotides with a non-strand displacing, non 5′-3′ exonuclease DNA polymerase.

In some embodiments, step (c) comprises ligation with a DNA ligase.

In some embodiments, the template polynucleotide comprises a barcode sequence and/or a UMI.

In some embodiments, the first and second oligonucleotides are about 10 to about 50 nucleotides in length. In some embodiments, the second and third sequences of the extension product to which the second oligonucleotide hybridizes are about 10 to about 50 nucleotides in length. In some embodiments, the second oligonucleotide comprises a linker between the sequences that are complementary to the second and third sequences of the extension product. The linker may be any nucleotide sequence, e.g., a sequence that is not complementary to a sequence of the extension product. In one embodiment, the linker comprises the sequence (AT)ₙ. In some embodiments, the linker is about 1 to about 50 nucleotides in length.

In some embodiments, the method further comprises: (d) determining all or a portion of the 5′ and 3′ portions of the template polynucleotide sequence (the sequences that are 5′ and 3′ to the fourth sequence of the template polynucleotide) or a complement thereof. Prior to step (d), the method may comprise: generating an amplification product from the polynucleotide product generated in step (c).

In another aspect, a method is provided for detecting a biological analyte within a biological sample, said method comprising: (a) contacting a biological sample with a probe, wherein: the probe comprises, in a 5′ to 3′ direction: a barcode and a capture domain, wherein a template polynucleotide sequence of a biological analyte within the biological sample hybridizes to the capture domain; (b) extending a 3′ end of the probe to generate an extension product that comprises a nucleotide sequence that is complementary to the template polynucleotide sequence, or a portion thereof; (c) hybridizing first and second oligonucleotides to the extension product, wherein: the first oligonucleotide comprises: a sequence that is complementary to a first sequence of the extension product; the second oligonucleotide is a bridging oligonucleotide that comprises: sequences that are complementary to second and third sequences that are flanking a polynucleotide region to be removed from the extension product, and a 5′ phosphate group, wherein the second and third sequences of the extension product are 3′ and 5′, respectively, to a fourth sequence that comprises the polynucleotide region to be removed from the extension product, and wherein the second, third, and fourth sequences of the extension product are complementary to sequences of the template polynucleotide sequence, and wherein the first sequence of the extension product is 3′ to the second sequence of the extension product; (d) extending 3′ ends of the first and second oligonucleotides, thereby producing extended first and second oligonucleotides, wherein extension of the first oligonucleotide ceases when the 5′ phosphate of the second oligonucleotide is reached, thereby resulting in a nick between the extended first and second oligonucleotides; and (e) ligating the 3′ end of the extended first oligonucleotide and the 5′ phosphate of the extended second oligonucleotide, thereby producing a polynucleotide product that comprises a complement of 5′ and 3′ portions of the extension product, wherein the polynucleotide product and does not comprise the complement of the fourth sequence of the extension product.

In some embodiments, the probe further comprises a UMI positioned 5′ to the capture domain.

In some embodiments, the first sequence of the extension product to which the first oligonucleotide hybridizes is at the 3′ end of the template polynucleotide.

In some embodiments, the first sequence of the extension product to which the first oligonucleotide hybridizes is an adapter that is attached to the 3′ end of the extension product. In one embodiment, prior to step (a): (i) untemplated nucleotides are added to the 3′ end of the extension product; a template switching oligonucleotide is hybridized to the untemplated nucleotides; and (ii) the 3′ end of the extension product is extended, thereby generating the adapter sequence, wherein the adapter sequence is complementary to the sequence of the template switching oligonucleotide. The untemplated nucleotides may comprise any nucleotides or sequence of nucleotides. In some embodiments, the untemplated nucleotides comprise a poly(C) or poly(G) sequence. In another embodiment, prior to step (a), the adapter is ligated to the 3′ end of the extension product.

In some embodiments, step (b) comprises extending the 3′ end of the probe with a RNA dependent or DNA dependent DNA polymerase.

In some embodiments, step (d) comprises extending the 3′ ends of the first and second oligonucleotides with a non-strand displacing, non 5′-3′ exonuclease DNA polymerase.

In some embodiments, step (e) comprises ligation with a DNA ligase.

In some embodiments, step (b) comprises incorporation of uridine residues into the extension product, and the method further comprises: (f) digesting the extension product with a uracil specific excision reagent.

In some embodiments, the first and second oligonucleotides are about 10 to about 50 nucleotides in length. In some embodiments, the second and third sequences of the extension product to which the second oligonucleotide hybridizes are about 10 to about 50 nucleotides in length. In some embodiments, the second oligonucleotide comprises a linker between the sequences that are complementary to the second and third sequences of the extension product. The linker may be any nucleotide sequence, e.g., a sequence that is not complementary to a sequence of the extension product. In one embodiment, the linker comprises the sequence (AT) n. In some embodiments, the linker is about 1 to about 50 nucleotides in length.

In some embodiments, the polynucleotide probe comprises a barcode sequence 5′ to the capture domain, for example, a cell barcode sequence or a spatial barcode sequence.

In some embodiments, wherein step (a) further comprises permeabilizing the biological sample prior to or after contacting the substrate.

In some embodiments, step (a) further comprises permeabilizing the biological sample prior to or after contacting the substrate.

In various embodiments, the biological sample may be a tissue section, a primary cell, a cell line, or an organoid. For example, the biological sample may be a formalin-fixed and paraffin-embedded (FFPE) tissue section or a fresh frozen tissue section.

The target polynucleotide may comprise DNA, RNA. The first capture domain may comprise a poly(dT) sequence, a random sequence, or a sequence that is complementary to a template polynucleotide sequence of interest. In one embodiment, the target polynucleotide comprises mRNA. For example, the mRNA may encode a T-cell receptor or B-cell receptor, and in one embodiment, the method further comprises determining a sequence that encodes one or more complementarity determining region(s) (CDR(s)) of the T-cell receptor or B-cell receptor. For example, the mRNA may encode an immunoglobulin heavy or light chain, and in one embodiment, the method further comprises determining a sequence that encodes one or more CDR(s) of the immunoglobulin heavy or light chain.

In some embodiments, the 5′ end of the probe is attached to the substrate. In some embodiments, the substrate comprises an array comprising a feature, wherein the 5′ end of the probe is attached to the feature. For example, the array may be a bead array (for example, gel beads) or a slide.

In some embodiment, the probe is directly attached to the substrate. In other embodiments, the probe is indirectly attached to the substrate. For example, the probe may be attached to beads and the beads attached to the substrate.

In some embodiments, the method further comprises: (f) determining (i) all or a portion of the 5′ and 3′ portions of the template polynucleotide sequence, or a complement thereof, and (ii) the sequence of all or a portion of a spatial barcode sequence, or a complement thereof, and using the determined sequences of (i) and (ii) to identify the location of the biological analyte in the biological sample. Prior to step (f), the method may further comprise: generating an amplification product from the polynucleotide product generated in step (e), and wherein step (f) comprises determining the sequences of (i) and (ii) using the amplification product.

In some embodiments, the present invention provides for kits that comprise arrays for practicing the methods described herein. The array of a kit can comprise a feature, where the feature comprises an attached first and second probe, wherein: a 5′ end of the first probe is attached to the feature; the first probe comprises in a 5′ to a 3′ direction: a spatial barcode and a poly(T) capture domain, where the poly(T) capture domain binds specifically to the target nucleic acid; a 5′ end of the second probe is attached to the feature; a 3′ end of the second probe is reversibly blocked; and the second probe comprises a poly(GI) capture domain. A kit can comprise enzymes, buffers, reagents, etc. for practicing the methods disclosed here. Further, a kit can include instructions such that users of the kit would understand how to use the kit to capture a target analyte (e.g. a nucleic acid) from a biological sample and determine its location.

All publications, patents, patent applications, and information available on the internet and mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “each,” when used in reference to a collection of items, is intended to identify an individual item in the collection but does not necessarily refer to every item in the collection, unless expressly stated otherwise, or unless the context of the usage clearly indicates otherwise.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### DESCRIPTION OF DRAWINGS

The following drawings illustrate certain embodiments of the features and advantages of this disclosure. These embodiments are not intended to limit the scope of the appended claims in any manner. Like reference symbols in the drawings indicate like elements.

FIG. 1 is a schematic diagram showing an example of a barcoded capture probe, as described herein.

FIG. 2 is a schematic diagram showing an exemplary feature comprising an attached first probe and second probe.

FIG. 3A-3C are workflow schematics illustrating exemplary steps for A) generating a spatially-barcoded sample for analysis and for use in further steps of the methods described herein (the first through the third step from the left show a captured nucleic acid comprising SEQ ID NO: 1), B) is a workflow schematic illustrating exemplary steps for generating a spatially-barcoded sample for analysis that allows for the sequencing of the target nucleic acid from both the 3′ end and the 5′ end (the second through the fourth steps, the sixth step, and the seventh step from the left show an extended first probe comprising SEQ ID NO: 2, and the fifth step from the left shows an extended first probe comprising SEQ ID NO: 3), and C) a schematic diagram showing an exemplary spatially-barcoded sample for analysis generated using the methods described herein.

FIGS. 4A-4J depict an exemplary workflow for detecting and/or determining spatial location of a target polynucleotide of interest.

FIG. 5 depicts an exemplary workflow for analysis of one or more analyte(s) from single cells. A captured nucleic acid comprising SEQ ID NO: 4 is shown.

FIG. 6 depicts an exemplary workflow for processing of a template polynucleotide to remove a sequence that is not of interest for detection and/or analysis.

### DETAILED DESCRIPTION

Provided herein are methods of determining a location of a target nucleic acid in a biological sample that include: (a) contacting the biological sample with an array comprising a feature, wherein the feature comprises an attached first and second probe, where: a 5′ end of the first probe is attached to the feature; the first probe comprises in a 5′ to a 3′ direction: a spatial barcode and a poly(T) capture domain, where the poly(T) capture domain binds specifically to the target nucleic acid; a 5′ end of the second probe is attached to the feature; a 3′ end of the second probe is reversibly blocked; and the second probe comprises a poly(GI) capture domain; (b) extending a 3′ end of the first probe to add a sequence that is complementary to a portion of the target nucleic acid; (c) ligating an adapter to the 5′ end of the target nucleic acid specifically bound to the first probe; (d) adding a sequence complementary to the adapter to the 3′ end of the first probe; (e) adding non-templated cytosines to the 3′ end of the first probe to generate a poly(C) sequence, where the poly(C) sequence specifically binds to the poly(GI) capture domain of the second probe; (f) unblocking the 3′ end of the second probe and extending the 3′ end of the second probe to add a sequence comprising a sequence in the target nucleic acid and a sequence that is complementary to the spatial barcode; (g) cleaving a region of the second probe at a cleavage site that is 5′ to the poly(GI) capture domain, thereby releasing the second probe from the feature; and (h) determining (i) all or a part of the sequence of the spatial barcode, or a complement thereof, and (ii) all or a part of the sequence of the target nucleic acid, or a complement thereof, and using the sequences of (i) and (ii) to determine the location of the target nucleic acid in the biological sample.

Also provided herein are arrays comprising a feature, where the feature comprises an attached first and second probe, wherein: a 5′ end of the first probe is attached to the feature; the first probe comprises in a 5′ to a 3′ direction: a spatial barcode and a poly(T) capture domain, where the poly(T) capture domain binds specifically to the target nucleic acid; a 5′ end of the second probe is attached to the feature; a 3′ end of the second probe is reversibly blocked; and the second probe comprises a poly(GI) capture domain.

### I. Introduction

Spatial analysis methodologies and compositions described herein can provide a vast amount of analyte and/or expression data for a variety of analytes within a biological sample at high spatial resolution, while retaining native spatial context. Spatial analysis methods and compositions can include, e.g., the use of a capture probe including a spatial barcode (e.g., a nucleic acid sequence that provides information as to the location or position of an analyte within a cell or a tissue sample (e.g., mammalian cell or a mammalian tissue sample) and a capture domain that is capable of binding to an analyte (e.g., a protein and/or a nucleic acid) produced by and/or present in a cell. Spatial analysis methods and compositions can also include the use of a capture probe having a capture domain that captures an intermediate agent for indirect detection of an analyte. For example, the intermediate agent can include a nucleic acid sequence (e.g., a barcode) associated with the intermediate agent. Detection of the intermediate agent is therefore indicative of the analyte in the cell or tissue sample.

Non-limiting aspects of spatial analysis methodologies and compositions are described in U.S. Pat. Nos. 10,774,374, 10,724,078, 10,480,022, 10,059,990, 10,041,949, 10,002,316, 9,879,313, 9,783,841, 9,727,810, 9,593,365, 8,951,726, 8,604,182, 7,709,198, U.S. Patent Application Publication Nos. 2020/239946, 2020/080136, 2020/0277663, 2020/024641, 2019/330617, 2019/264268, 2020/256867, 2020/224244, 2019/194709, 2019/161796, 2019/085383, 2019/055594, 2018/216161, 2018/051322, 2018/0245142, 2017/241911, 2017/089811, 2017/067096, 2017/029875, 2017/0016053, 2016/108458, 2015/000854, 2013/171621, WO 2018/091676, WO 2020/176788, Rodriques et al., Science 363(6434):1463-1467,2019; Lee et al., Nat. Protoc. 10(3):442-458, 2015; Trejo et al., PLoS ONE 14(2):c0212031, 2019; Chen et al., Science 348(6233):aaa6090, 2015; Gao et al., BMC Biol. 15:50, 2017; and Gupta et al., Nature Biotechnol. 36:1197-1202, 2018; the Visium Spatial Gene

Expression Reagent Kits User Guide (e.g., Rev C, dated June 2020), and/or the Visium Spatial Tissue Optimization Reagent Kits User Guide (e.g., Rev C, dated July 2020), both of which are available at the 10× Genomics Support Documentation website, and can be used herein in any combination. Further non-limiting aspects of spatial analysis methodologies and compositions are described herein.

Some general terminology that may be used in this disclosure can be found in Section (I)(b) of WO 2020/176788 and/or U.S. Patent Application Publication No. 2020/0277663. Typically, a “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. For the purpose of this disclosure, an “analyte” can include any biological substance, structure, moiety, or component to be analyzed. The term “target” can similarly refer to an analyte of interest.

Analytes can be broadly classified into one of two groups: nucleic acid analytes, and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In some embodiments, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some embodiments, analyte(s) can be peptides or proteins, including without limitation antibodies and enzymes. Additional examples of analytes can be found in Section (I)(c) of WO 2020/176788 and/or U.S. Patent Application Publication No. 2020/0277663. In some embodiments, an analyte can be detected indirectly, such as through detection of an intermediate agent, for example, a ligation product or an analyte capture agent (e.g., an oligonucleotide-conjugated antibody), such as those described herein.

A “biological sample” is typically obtained from the subject for analysis using any of a variety of techniques including, but not limited to, biopsy, surgery, and laser capture microscopy (LCM), and generally includes cells and/or other biological material from the subject. In some embodiments, a biological sample can be a tissue section. In some embodiments, a biological sample can be a fixed and/or stained biological sample (e.g., a fixed and/or stained tissue section). Non-limiting examples of stains include histological stains (e.g., hematoxylin and/or eosin) and immunological stains (e.g., fluorescent stains). In some embodiments, a biological sample (e.g., a fixed and/or stained biological sample) can be imaged. In some embodiments, a biological sample can be a blood sample, a tumor sample, a lymph node sample, or a thymus sample. Biological samples are also described in Section (I)(d) of WO 2020/176788 and/or U.S. Patent Application Publication No. 2020/0277663.

In some embodiments, a biological sample is permeabilized with one or more permeabilization reagents. For example, permeabilization of a biological sample can facilitate analyte capture. Exemplary permeabilization agents and conditions are described in Section (I)(d)(ii)(13) or the Exemplary Embodiments Section of WO 2020/176788 and/or U.S. Patent Application Publication No. 2020/0277663.

Array-based spatial analysis methods involve the transfer of one or more analytes from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI)) and a capture domain). In some embodiments, a capture probe can include a cleavage domain and/or a functional domain (e.g., a primer-binding site, such as for next-generation sequencing (NGS)). See, e.g., Section (II)(b) (e.g., subsections (i)-(vi)) of WO 2020/176788 and/or U.S. Patent Application Publication No. 2020/0277663. Generation of capture probes can be achieved by any appropriate method, including those described in Section (II)(d)(ii) of WO 2020/176788 and/or U.S. Patent Application Publication No. 2020/0277663.

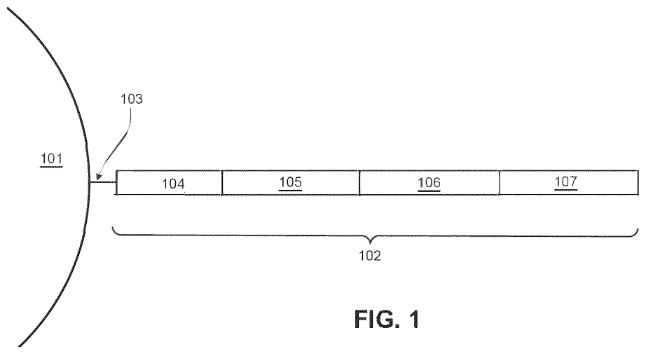

FIG. 1 is a schematic diagram showing an exemplary capture probe, as described herein. As shown, the capture probe 102 is optionally coupled to a feature 101 by a cleavage domain 103, such as a disulfide linker. The capture probe can include a functional sequence 104 that are useful for subsequent processing. The functional sequence 104 can include all or a part of sequencer specific flow cell attachment sequence (e.g., a P5 or P7 sequence), all or a part of a sequencing primer sequence, (e.g., a R1 primer binding site, a R2 primer binding site), or combinations thereof. The capture probe can also include a spatial barcode 105. The capture probe can also include a unique molecular identifier (UMI) sequence 106. While FIG. 1 shows the spatial barcode 105 as being located upstream (5′) of UMI sequence 106, it is to be understood that capture probes wherein UMI sequence 106 is located upstream (5′) of the spatial barcode 105 is also suitable for use in any of the methods described herein. The capture probe can also include a capture domain 107 to facilitate capture of a target analyte. In some embodiments, the capture probe comprises one or more additional functional sequences that can be located, for example between the spatial barcode 105 and the UMI sequence 106, between the UMI sequence 106 and the capture domain 107, or following the capture domain 107. The capture domain can have a sequence complementary to a sequence of a nucleic acid analyte. The capture domain can have a sequence complementary to a connected probe described herein. The capture domain can have a sequence complementary to a capture handle sequence present in an analyte capture agent. The capture domain can have a sequence complementary to a splint oligonucleotide. Such splint oligonucleotide, in addition to having a sequence complementary to a capture domain of a capture probe, can have a sequence of a nucleic acid analyte, a sequence complementary to a portion of a connected probe described herein, and/or a capture handle sequence described herein.

The functional sequences can generally be selected for compatibility with any of a variety of different sequencing systems, e.g., Ion Torrent Proton or PGM, Illumina sequencing instruments, PacBio, Oxford Nanopore, etc., and the requirements thereof. In some embodiments, functional sequences can be selected for compatibility with non-commercialized sequencing systems. Examples of such sequencing systems and techniques, for which suitable functional sequences can be used, include (but are not limited to) Ion Torrent Proton or PGM sequencing, Illumina sequencing, PacBio SMRT sequencing, and Oxford Nanopore sequencing. Further, in some embodiments, functional sequences can be selected for compatibility with other sequencing systems, including non-commercialized sequencing systems.

In some embodiments, the spatial barcode 105 and functional sequences 104 is common to all of the probes attached to a given feature. In some embodiments, the UMI sequence 106 of a capture probe attached to a given feature is different from the UMI sequence of a different capture probe attached to the given feature.

In some embodiments, more than one analyte type (e.g., nucleic acids and proteins) from a biological sample can be detected (e.g., simultaneously or sequentially) using any appropriate multiplexing technique, such as those described in Section (IV) of WO 2020/176788 and/or U.S. Patent Application Publication No. 2020/0277663.

In some embodiments, detection of one or more analytes (e.g., protein analytes) can be performed using one or more analyte capture agents. As used herein, an “analyte capture agent” refers to an agent that interacts with an analyte (e.g., an analyte in a biological sample) and with a capture probe (e.g., a capture probe attached to a substrate or a feature) to identify the analyte. In some embodiments, the analyte capture agent includes: (i) an analyte binding moiety (e.g., that binds to an analyte), for example, an antibody or antigen-binding fragment thereof; (ii) analyte binding moiety barcode; and (iii) an analyte capture sequence. As used herein, the term “analyte binding moiety barcode” refers to a barcode that is associated with or otherwise identifies the analyte binding moiety. As used herein, the term “analyte capture sequence” refers to a region or moiety configured to hybridize to, bind to, couple to, or otherwise interact with a capture domain of a capture probe. In some cases, an analyte binding moiety barcode (or portion thereof) may be able to be removed (e.g., cleaved) from the analyte capture agent. Additional description of analyte capture agents can be found in Section (II)(b)(ix) of WO 2020/176788 and/or Section (II) (b) (viii) U.S. Patent Application Publication No. 2020/0277663.

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) out of a cell and towards a spatially-barcoded array (e.g., including spatially-barcoded capture probes). Another method is to cleave spatially-barcoded capture probes from an array and promote the spatially-barcoded capture probes towards and/or into or onto the biological sample.

In some cases, capture probes may be configured to prime, replicate, and consequently yield optionally barcoded extension products from a template (e.g., a DNA or RNA template, such as an analyte or an intermediate agent (e.g., a ligation product or an analyte capture agent), or a portion thereof), or derivatives thereof (see, e.g., Section (II)(b)(vii) of WO 2020/176788 and/or U.S. Patent Application Publication No. 2020/0277663 regarding extended capture probes). In some cases, capture probes may be configured to form ligation products with a template (e.g., a DNA or RNA template, such as an analyte or an intermediate agent, or portion thereof), thereby creating ligations products that serve as proxies for a template.

As used herein, an “extended capture probe” refers to a capture probe having additional nucleotides added to the terminus (e.g., 3′ or 5′ end) of the capture probe thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes adding to a 3′ end of a capture probe a nucleic acid sequence that is complementary to a nucleic acid sequence of an analyte or intermediate agent specifically bound to the capture domain of the capture probe. In some embodiments, the capture probe is extended using reverse transcription. In some embodiments, the capture probe is extended using one or more DNA polymerases. The extended capture probes include the sequence of the capture probe and the sequence of the spatial barcode of the capture probe.

In some embodiments, extended capture probes are amplified (e.g., in bulk solution or on the array) to yield quantities that are sufficient for downstream analysis, e.g., via DNA sequencing. In some embodiments, extended capture probes (e.g., DNA molecules) act as templates for an amplification reaction (e.g., a polymerase chain reaction).

Additional variants of spatial analysis methods, including in some embodiments, an imaging step, are described in Section (II)(a) of WO 2020/176788 and/or U.S. Patent Application Publication No. 2020/0277663. Analysis of captured analytes (and/or intermediate agents or portions thereof), for example, including sample removal, extension of capture probes, sequencing (e.g., of a cleaved extended capture probe and/or a cDNA molecule complementary to an extended capture probe), sequencing on the array (e.g., using, for example, in situ hybridization or in situ ligation approaches), temporal analysis, and/or proximity capture, is described in Section (II)(g) of WO 2020/176788 and/or U.S. Patent Application Publication No. 2020/0277663. Some quality control measures are described in Section (II)(h) of WO 2020/176788 and/or U.S. Patent Application Publication No. 2020/0277663.

Spatial information can provide information of biological and/or medical importance. For example, the methods and compositions described herein can allow for: identification of one or more biomarkers (e.g., diagnostic, prognostic, and/or for determination of efficacy of a treatment) of a disease or disorder; identification of a candidate drug target for treatment of a disease or disorder; identification (e.g., diagnosis) of a subject as having a disease or disorder; identification of stage and/or prognosis of a disease or disorder in a subject; identification of a subject as having an increased likelihood of developing a disease or disorder; monitoring of progression of a disease or disorder in a subject; determination of efficacy of a treatment of a disease or disorder in a subject; identification of a patient subpopulation for which a treatment is effective for a disease or disorder; modification of a treatment of a subject with a disease or disorder; selection of a subject for participation in a clinical trial; and/or selection of a treatment for a subject with a disease or disorder.

Spatial information can provide information of biological importance. For example, the methods and compositions described herein can allow for: identification of transcriptome and/or proteome expression profiles (e.g., in healthy and/or diseased tissue); identification of multiple analyte types in close proximity (e.g., nearest neighbor analysis); determination of up-and/or down-regulated genes and/or proteins in diseased tissue; characterization of tumor microenvironments; characterization of tumor immune responses; characterization of cells types and their co-localization in tissue; and identification of genetic variants within tissues (e.g., based on gene and/or protein expression profiles associated with specific disease or disorder biomarkers).

Typically, for spatial array-based methods, a substrate functions as a support for direct or indirect attachment of capture probes to features of the array. A “feature” is an entity that acts as a support or repository for various molecular entities used in spatial analysis. In some embodiments, some or all of the features in an array are functionalized for analyte capture. Exemplary substrates are described in Section (II)(c) of WO 2020/176788 and/or U.S. Patent

Application Publication No. 2020/0277663. Exemplary features and geometric attributes of an array can be found in Sections (II)(d)(i), (II)(d)(iii), and (II)(d)(iv) of WO 2020/176788 and/or U.S. Patent Application Publication No. 2020/0277663.

Generally, analytes and/or intermediate agents (or portions thereof) can be captured when contacting a biological sample with a substrate including capture probes (e.g., a substrate with capture probes embedded, spotted, printed, fabricated on the substrate, or a substrate with features (e.g., beads, wells) comprising capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in Section (II)(e) of WO 2020/176788 and/or U.S. Patent Application Publication No. 2020/0277663.

In some cases, spatial analysis can be performed by attaching and/or introducing a molecule (e.g., a peptide, a lipid, or a nucleic acid molecule) having a barcode (e.g., a spatial barcode) to a biological sample (e.g., to a cell in a biological sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some embodiments, after attaching and/or introducing a molecule having a barcode to a biological sample, the biological sample can be physically separated (e.g., dissociated) into single cells or cell groups for analysis. Some such methods of spatial analysis are described in Section (III) of WO 2020/176788 and/or U.S. Patent Application Publication No. 2020/0277663.

In some cases, spatial analysis can be performed by detecting multiple oligonucleotides that hybridize to an analyte. In some instances, for example, spatial analysis can be performed using RNA-templated ligation (RTL). Methods of RTL have been described previously. See, e.g., Credle et al., Nucleic Acids Res. 2017 Aug. 21; 45(14):e128. Typically, RTL includes hybridization of two oligonucleotides to adjacent sequences on an analyte (e.g., an RNA molecule, such as an mRNA molecule). In some instances, the oligonucleotides are DNA molecules. In some instances, one of the oligonucleotides includes at least two ribonucleic acid bases at the 3′ end and/or the other oligonucleotide includes a phosphorylated nucleotide at the 5′ end. In some instances, one of the two oligonucleotides includes a capture domain (e.g., a poly(A) sequence, a non-homopolymeric sequence). After hybridization to the analyte, a ligase (e.g., SplintR ligase) ligates the two oligonucleotides together, creating a ligation product. In some instances, the two oligonucleotides hybridize to sequences that are not adjacent to one another. For example, hybridization of the two oligonucleotides creates a gap between the hybridized oligonucleotides. In some instances, a polymerase (e.g., a DNA polymerase) can extend one of the oligonucleotides prior to ligation. After ligation, the ligation product is released from the analyte. In some instances, the ligation product is released using an endonuclease (e.g., RNAse H). The released ligation product can then be captured by capture probes (e.g., instead of direct capture of an analyte) on an array, optionally amplified, and sequenced, thus determining the location and optionally the abundance of the analyte in the biological sample.

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the analytes they capture are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that spatial barcodes are uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location, a fiducial marker) for the array. Accordingly, each feature location has an “address” or location in the coordinate space of the array.

Some exemplary spatial analysis workflows are described in the Exemplary Embodiments section of WO 2020/176788 and/or U.S. Patent Application Publication No. 2020/0277663. See, for example, the Exemplary embodiment starting with “In some non-limiting examples of the workflows described herein, the sample can be immersed . . . ” of WO 2020/176788 and/or U.S. Patent Application Publication No. 2020/0277663. See also, e.g., the Visium Spatial Gene Expression Reagent Kits User Guide (e.g., Rev C, dated June 2020), and/or the Visium Spatial Tissue Optimization Reagent Kits User Guide (e.g., Rev C, dated July 2020).

In some embodiments, spatial analysis can be performed using dedicated hardware and/or software, such as any of the systems described in Sections (II)(e)(ii) and/or (V) of WO 2020/176788 and/or U.S. Patent Application Publication No. 2020/0277663, or any of one or more of the devices or methods described in Sections Control Slide for Imaging, Methods of Using Control Slides and Substrates for, Systems of Using Control Slides and Substrates for Imaging, and/or Sample and Array Alignment Devices and Methods, Informational labels of WO 2020/123320.

Suitable systems for performing spatial analysis can include components such as a chamber (e.g., a flow cell or scalable, fluid-tight chamber) for containing a biological sample. The biological sample can be mounted for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid state storage medium such as, but not limited to, a magnetic, optical, or other solid state, persistent, writeable and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD, CMOS) used to capture images. The systems can also optionally include one or more light sources (e.g., LED-based, diode-based, lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein.

In some cases, the systems described herein can detect (e.g., register an image) the biological sample on the array. Exemplary methods to detect the biological sample on an array are described in PCT Application No. 2020/061064 and/or U.S. patent application Ser. No. 16/951,854.

Prior to transferring analytes from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Exemplary methods to generate a two-and/or three-dimensional map of the analyte presence and/or level are described in PCT Application No. 2020/053655 and spatial analysis methods are generally described in WO 2020/061108 and/or U.S. patent application Ser. No. 16/951,864.

In some cases, a map of analyte presence and/or level can be aligned to an image of a biological sample using one or more fiducial markers, e.g., objects placed in the field of view of an imaging system which appear in the image produced, as described in the Substrate Attributes Section, Control Slide for Imaging Section of WO 2020/123320, PCT Application No. 2020/061066, and/or U.S. patent application Ser. No. 16/951,843. Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distances.

### II. Spatial 5′ Gene Expression of VDJ Libraries

Provided herein are methods of determining a location of a target nucleic acid in a biological sample that include: (a) contacting the biological sample with an array comprising a feature, where the feature comprises an attached first and second probe, wherein: a 5′ end of the first probe is attached to the feature; the first probe comprises in a 5′ to a 3′ direction: a spatial barcode and a poly(T) capture domain, where the poly(T) capture domain binds specifically to the target nucleic acid; a 5′ end of the second probe is attached to the feature; a 3′ end of the second probe is reversibly blocked; and the second probe comprises a poly(GI) capture domain; (b) extending a 3′ end of the first probe to add a sequence that is complementary to a portion of the target nucleic acid; (c) ligating an adapter to the 5′ end of the target nucleic acid specifically bound to the first probe; (d) adding a sequence complementary to the adapter to the 3′ end of the first probe; (e) adding non-templated cytosines to the 3′ end of the first probe to generate a poly(C) sequence, where the poly(C) sequence specifically binds to the poly(GI) capture domain of the second probe; (f) unblocking the 3′ end of the second probe and extending the 3′ end of the second probe to add a sequence comprising a sequence in the target nucleic acid and a sequence that is complementary to the spatial barcode; (g) cleaving a region of the second probe at a cleavage site that is 5′ to the poly(GI) capture domain, thereby releasing the second probe from the feature; and (h) determining (i) all or a part of the sequence of the spatial barcode, or a complement thereof, and (ii) all or a part of the sequence of the target nucleic acid, or a complement thereof, and using the sequences of (i) and (ii) to determine the location of the target nucleic acid in the biological sample. In some embodiments, a feature can include two or more pairs of a first and a second probe (e.g., any of the first and second probes described herein). A first pair of a first and a second probe at a feature, as compared to a second pair of a first and a second probe at the feature, can have a different first and/or second probe as compared to first and/or second probe of the second pair (e.g., a different capture domain in the first probe and/or a different barcode in the first and/or second probes). In some embodiments, the spatial barcode in the first probe of the first pair and the spatial barcode in the first probe of the second pair are the same. In some embodiments, the spatial barcode in the first probe of the first pair and the spatial barcode in the first probe of the second pair are different. In some embodiments, the capture domain of the first probe of the first pair is the same as the capture domain of the first probe of the second pair. In some embodiments, the capture domain of the first probe of the first pair is different from the capture domain of the first probe of the second pair.

In some embodiments, the capture domain on the first probe has a poly(T) capture domain, where the poly(T) capture domain is configured to interact with the target nucleic acid (e.g., positioned at the 3′ end of the first probe). For example, the poly(T) capture domain specifically binds to a messenger RNA (mRNA), via the poly(A) tail of the mRNA. For example, a poly(T) capture domain can include at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 contiguous thymidines.

In some embodiments, the poly(GI) capture domain of the second probe is configured to interact with a poly(C) tail of an oligonucleotide, e.g., a poly(C) tail added to the 3′ end of the extended first probe. In some embodiments, the poly(C) tail is added to the 3′ end of the first probe after the extension of the first probe to add a sequence that is complementary to a portion of the target nucleic acid. In some embodiments, the poly(GI) capture domain comprises a sequence of at least 5 contiguous guanosine(s) and/or inosine(s). For example, a poly(GI) capture domain comprises a sequence of (GGI)n, wherein n is about 3 to about 20. In some embodiments, the poly(GI) capture domain comprises a sequence of (GGI)n, wherein n is 1, 2, 3, 4, 5, 6, 7, 8, 9, 10, 11, 12, 13, 14, 15, 16, 17, 18, 19, or 20. For example, a poly(GI) capture domain comprises a sequence of (GI)n, wherein n is about 4 to about 30. For example, a poly(GI) capture domain comprises a sequence of (GI)n, wherein n is 4, 5, 6, 7, 8, 9, 10, 11, 12, 13, 14, 15, 16, 17, 18, 19, 20, 21, 22, 23, 24, 25, 26, 27, 28, 29, or 30. For example, a poly(GI) capture domain comprises a sequence of (IG)n, wherein n is about 4 to about 30. For example, a poly(GI) capture domain comprises a sequence of (IG)n, wherein n is 4, 5, 6, 7, 8, 9, 10, 11, 12, 13, 14, 15, 16, 17, 18, 19, 20, 21, 22, 23, 24, 25, 26, 27, 28, 29, or 30.

In some embodiments, the second probe can comprise a spatial barcode, which is positioned 5′ to the poly(GI) capture domain. In some embodiments, the spatial barcode in the first probe is different from the spatial barcode sequence in the second probe. In some embodiments, the spatial barcode in the first probe is the same as the spatial barcode sequence in the second probe.

In some embodiments, both the first and the second probes are cleavable. In some embodiments, the first probe and the second probe have different cleavage sites and are cleavable using different methods. In some embodiments, the first probe and the second probe have the same cleavable site and are cleavable using the same method. In some embodiments, the cleavage domain of the first probe is 5′ to the poly(T) capture domain and/or the cleavage domain of the second probe is 5′ to the poly(GI) capture domain.

In some embodiments, the first probe is not cleavable and the second probe is cleavable. In some embodiments, the cleavage site of the second probe is 5′ to the poly(GI) capture domain of the second probe. In some embodiments, the cleavage site on the second probe is a uracil. In some embodiments, the uracil is cleaved by USER (Uracil-Specific Excision Reagent).

In some embodiments, the first probe further comprises a unique molecular identifier (UMI). In some embodiments, the second probe further comprises a unique molecular identifier (UMI). In some embodiments, the UMI in the first probe and the UMI in the second probe comprise different sequences. In some embodiments, the UMI in the first probe and the UMI in the second probe comprise the same sequence.

The 3′ end of the second probe can be blocked by chemical modification, e.g., addition of an azidomethyl group as a chemically reversible capping moiety such that the probe does not include a free 3′ end. Blocking or modifying the second probe, particularly at the free 3′ end of the capture domain, prior to contacting the biological sample with the array, prevents modification of the second probe, e.g., prevents the addition of a poly(A) tail to the free 3′ end of the second probe. Non-limiting examples of 3′ modifications include dideoxy C-3′ (3′-ddC), 3′ inverted dT, 3′ C3 spacer, 3′Amino, and 3′ phosphorylation.

In some embodiments, the second probe includes a restriction endonuclease recognition sequence or a sequence of nucleotides cleavable by specific enzyme activities. For example, uracil sequences can be enzymatically cleaved from a nucleotide sequence using uracil DNA glycosylase (UDG) or Uracil Specific Excision Reagent (USER). As another example, other modified bases (e.g., modified by methylation) can be recognized and cleaved by specific endonucleases. The second probe can be subjected to an enzymatic cleavage, which removes the blocking domain and any of the additional nucleotides that are added to the 3′ end of the capture probe during the modification process. Removal of the blocking domain reveals and/or restores the free 3′ end of the second probe. In some embodiments, additional nucleotides can be removed to reveal and/or restore the 3′ end of the second probe.

In some embodiments, a blocking domain can be incorporated into the second probe when it is synthesized, or after its synthesis. The terminal nucleotide of the capture domain is a reversible terminator nucleotide (e.g., 3′-O-blocked reversible terminator and 3′-unblocked reversible terminator), and can be included in the capture probe during or after probe synthesis.

“Adapter” refers to species that can be coupled to a polynucleotide sequence using any one of many different techniques including (but not limited to) ligation, hybridization, and tagmentation. Adaptors can also be nucleic acid sequences that add a function, e.g., spacer sequences, primer sequences/sites, barcode sequences, unique molecular identifier sequences. An adapter can include a sequencing primer sequence (e.g., R1 or a partial R1 (“pR1”), R2), or a flow cell attachment sequence (e.g., P5 or P7 or partial sequences thereof)).

Some embodiments of any of the methods described herein, step (h) includes sequencing all or a part of the sequence of the spatial barcode, or a complement thereof, and sequencing all of a part of the sequence of the target nucleic acid, or a complement thereof. The sequencing can be performed using any of the aforementioned methods. In some embodiments, step (h) includes sequencing the full-length sequence of the spatial barcode, or a complement thereof. In some embodiments, step (h) includes sequencing a part of the sequence of the spatial barcode, or a complement thereof. In some embodiments, step (h) includes sequencing the full-length sequence of the target nucleic acid, or a complement thereof. In some embodiments, step (h) includes sequencing a part of the target nucleic acid, or a complement thereof. In some embodiments, the sequencing is performed using high throughput sequencing. In some embodiments, the target nucleic acid is sequenced from the 5′ end of the target nucleic acid. In some embodiments, the target nucleic acid is sequenced from the 3′ end of the target nucleic acid. In some embodiments, the target nucleic acid is sequenced from both the 3′ end and the 5′ end of the target nucleic acid.

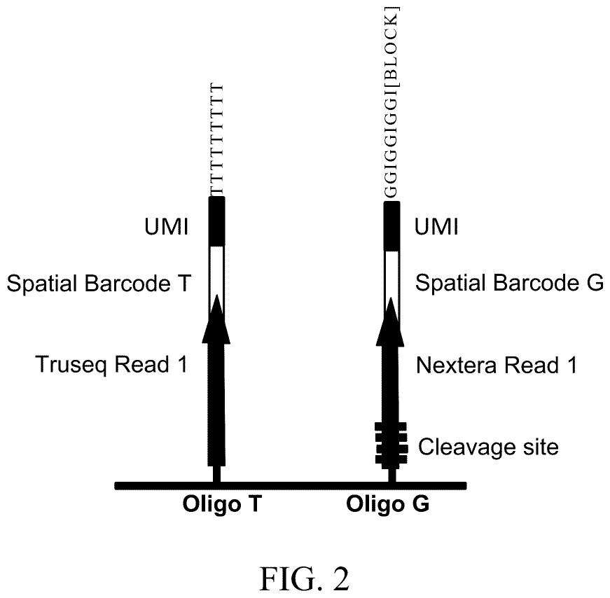

FIG. 2 is a schematic diagram showing an exemplary feature comprising an attached first and second probe. The first probe comprises in a 5′ to 3′ direction: a functional domain comprising a Truseq Read 1 primer, a spatial barcode, a UMI, and a poly(T) capture domain, where the poly(T) capture domain binds specifically to the target nucleic acid. The 5′ end of the first probe is attached to the feature.

The second probe comprises in a 5′ to 3′ direction: a cleavage domain, a functional domain comprising a Nextera Read 1 primer, a spatial barcode, a UMI, and a poly(GI) capture domain. The 5′ end of the second probe is attached to the feature. In some embodiments, the poly(GI) capture domain comprises a sequence of (GGI)n, wherein n is about 3 to about 20. In some embodiments, the poly(GI) capture domain comprises a sequence of (GGI)n, wherein n is 1, 2, 3, 4, 5, 6, 7, 8, 9, 10, 11, 12, 13, 14, 15, 16, 17, 18, 19, or 20. In some embodiments, the 3′ end of the second probe is reversibly blocked. The TruSeq and Nextera Read 1 primer sequences are used in Illumina sequencing workflows. However, it is understood that the present invention is not limited to any particular sequencing system, as such any primer or other functional sequences useful for other sequencing systems, as cited herein, can be equally used.

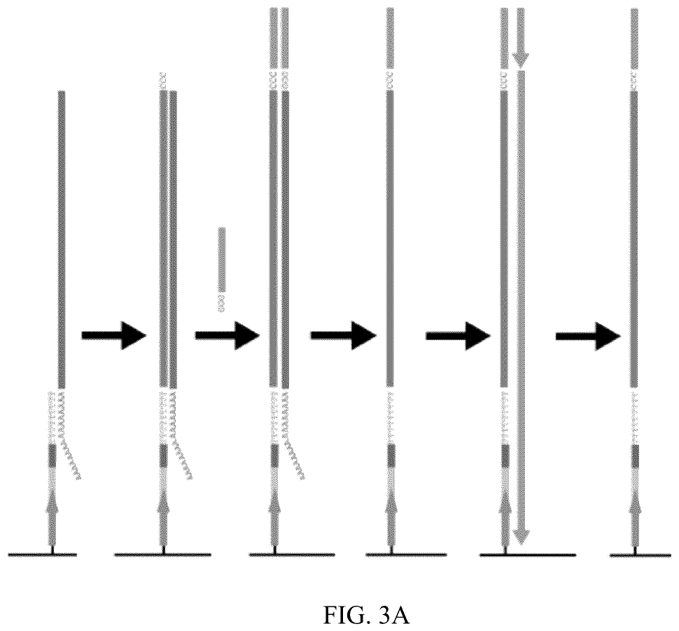

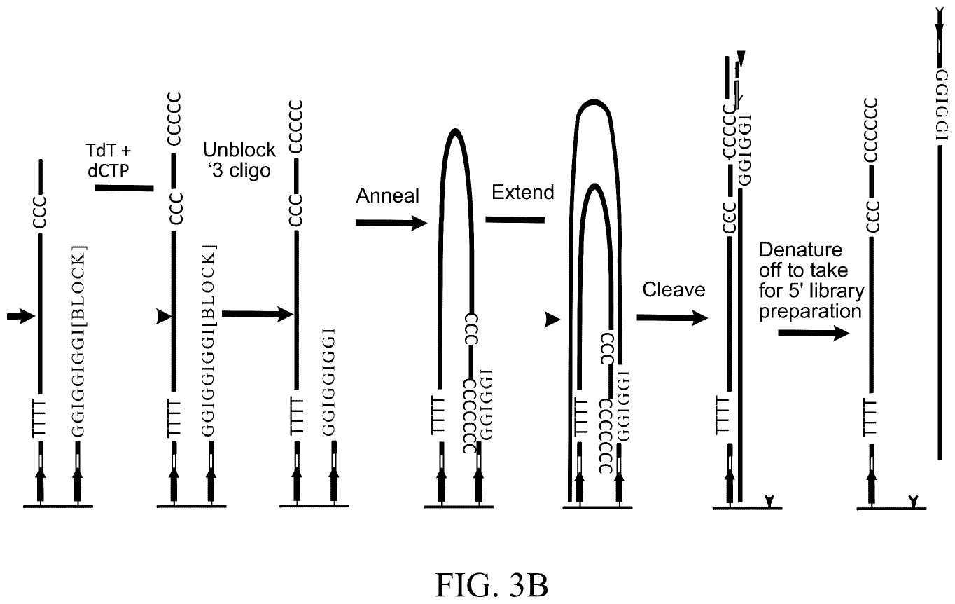

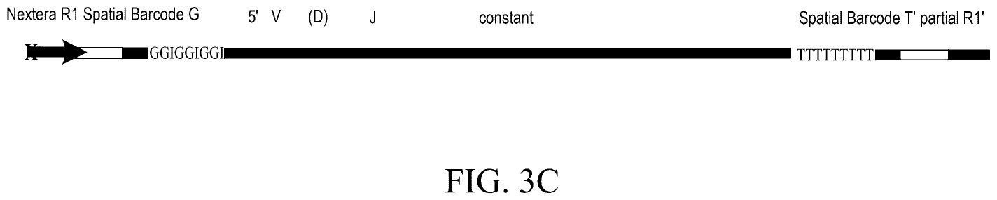

FIG. 3A is an exemplary diagram showing, from left to right, the annealing of the target analyte (e.g., target nucleic acid) to the poly(T) capture domain of the first probe; the extension of the first probe to add a sequence that is complementary to a portion of the target nucleic acid; the ligation of an adaptor to the 5′ end of the target nucleic acid specifically bound to the first probe; the addition of a sequence complementary to the adaptor to the 3′ end of the first probe; the releasing of the target nucleic acid from the first probe; the generation of a complement of the extended first probe; and the releasing of the complement of the extended first probe. In some embodiments, the released target nucleic acid is sequenced. In some embodiments, the released complement of the extended first probe is sequenced.

FIG. 3B is an exemplary diagram showing from left to right, the addition of non-templated cytosines to the 3′ end of the extended first probe (e.g., extended to include a sequence that is complementary to a portion of the sequence of a target nucleic acid, and optionally, further comprising an adaptor sequence or a functional domain) to generate a poly(C) sequence, where the poly(C) sequence specifically binds to the poly(GI) capture domain of the second probe; the unblocking of the 3′ end of the second probe; the hybridizing the poly(C) sequence on the first probe to the poly(GI) capture domain on the second capture probe; the extension of the 3′ end of the second probe to add a sequence complementary to the extended first capture probe. The final step is the releasing of the extended second probe sequence from the feature. In alternative embodiments, the second probe comprises a poly(T) capture domain and a poly(A) sequence is added to the 3′ end of the extended first probe (e.g., extended to add a sequence that is complementary to a portion of the sequence of a target nucleic acid), and the poly(A) sequence hybridizes to the poly(T) capture domain of the second probe.

FIG. 3C is a schematic diagram showing an example of a sequence generated by the methods described herein. The exemplary sequence shown comprises, from 5′ end to 3′ end, the functional domain of the second probe, which comprises a sequencing primer; the spatial barcode of the second probe; the UMI sequence of the second probe; the poly(GI) sequence of the second probe; the target nucleic acid sequence (from 5′ end to 3′ end); a sequence complementary to the UMI sequence of the first probe; a sequence complementary to the spatial barcode of the first probe; and a sequence complementary to part or the full sequence of the functional domain of the first probe, which comprises a sequencing primer. In some embodiments, the two sequencing primers have the same sequence. In some embodiments, the two sequencing primers have different sequences.

Further steps of the methods described herein include, for example, determining (i) all or a part of the sequence of the spatial barcode on either end of the sequence depicted in FIG. 3C, or a complement thereof, and (ii) all or a part of the sequence of the target nucleic acid, or a complement thereof, and using the sequences of (i) and (ii) to determine the location of the target nucleic acid in the biological samples.

The methods described herein allows for the sequencing of the target nucleic acid from either the 3′ end or the 5′ end, or both the 3′ and the 5′ ends of the target nucleic acid. For target nucleic acids that have large sizes (e.g., larger than 1 kb), the methods allow more accurate spatial sequence information to be obtained.

### III. Arrays for Making 5′ Libraries or VDJ Libraries

Also described herein is an array comprising a feature, where the feature comprises an attached first and second probe, wherein: a 5′ end of the first probe is attached to the feature; the first probe comprises in a 5′ to a 3′ direction: a spatial barcode and a poly(T) capture domain, wherein the poly(T) capture domain binds specifically to the target nucleic acid; a 5′ end of the second probe is attached to the feature; a 3′ end of the second probe is reversibly blocked; and the second probe comprises a poly(GI) capture domain.

In some embodiments of any of the arrays described herein, a feature can include two or more pairs of a first and a second probe (e.g., any of the first and second probes described herein). A first pair of a first and a second probe at a feature, as compared to a second pair of a first and a second probe at the feature, can have a different first and/or second probe as compared to first and/or second probe of the second pair (e.g., a different capture domain in the first probe and/or a different barcode in the first and/or second probes). In some embodiments of any of the arrays described herein, the spatial barcode in the first probe of the first pair and the spatial barcode in the first probe of the second pair are the same. In some embodiments of any of the arrays described herein, the spatial barcode in the first probe of the first pair and the spatial barcode in the first probe of the second pair are different. In some embodiments of any of the arrays described herein, the capture domain of the first probe of the first pair is the same as the capture domain of the first probe of the second pair. In some embodiments of any of the arrays described herein, the capture domain of the first probe of the first pair is different from the capture domain of the first probe of the second pair.

In some embodiments of any of the arrays described herein, the capture domain on the first probe has a poly(T) capture domain, where the poly(T) capture domain is configured to interact with a target nucleic acid (e.g., positioned at the 3′ end of the first probe). For example, the poly(T) capture domain specifically binds to a messenger RNA (mRNA), via the poly(A) tail of the mRNA. For example, a poly(T) capture domain can include at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 contiguous thymidines.

In some embodiments of any of the arrays described herein, the poly(GI) capture domain comprises a sequence of at least 5 contiguous guanosine(s) and/or inosine(s). For example, a poly(GI) capture domain comprises a sequence of (GGI)n, wherein n is about 3 to about 20. In some embodiments, the poly(GI) capture domain comprises a sequence of (GGI)n, wherein n is 1, 2, 3, 4, 5, 6, 7, 8, 9, 10, 11, 12, 13, 14, 15, 16, 17, 18, 19, or 20. For example, a poly(GI) capture domain comprises a sequence of (GI)n, wherein n is about 4 to about 30. For example, a poly(GI) capture domain comprises a sequence of (GI)n, wherein n is 4, 5, 6, 7, 8, 9, 10, 11, 12, 13, 14, 15, 16, 17, 18, 19, 20, 21, 22, 23, 24, 25, 26, 27, 28, 29, or 30. For example, a poly(GI) capture domain comprises a sequence of (GI)n, wherein n is about 4 to about 30. For example, a poly(GI) capture domain comprises a sequence of (GI)n, wherein n is 4, 5, 6, 7, 8, 9, 10, 11, 12, 13, 14, 15, 16, 17, 18, 19, 20, 21, 22, 23, 24, 25, 26, 27, 28, 29, or 30.

In some embodiments of any of the arrays described herein, the second probe can comprise a spatial barcode, which is positioned 5′ to the poly(GI) capture domain. In some embodiments, the spatial barcode in the first probe is different from the spatial barcode sequence in the second probe. In some embodiments, the spatial barcode in the first probe is the same as the spatial barcode sequence in the second probe.

In some embodiments of any of the arrays described herein, both the first and the second probes are cleavable. In some embodiments, the first probe and the second probe have different cleavage sites and are cleavable using different methods. In some embodiments, the first probe and the second probe have the same cleavable site and are cleavable using the same method. In some embodiments, the cleavage domain of the first probe is 5′ to the poly(T) capture domain and/or the cleavage domain of the second probe is 5′ to the poly(GI) capture domain.

In some embodiments of any of the arrays described herein, the first probe is not cleavable and the second probe is cleavable. In some embodiments, the cleavage site of the second probe is 5′ to the poly(GI) capture domain of the second probe. In some embodiments, the cleavage site on the second probe is a uracil. In some embodiments, the uracil is cleaved by USER (Uracil-Specific Excision Reagent).

In some embodiments of any of the arrays described herein, the first probe further comprises a unique molecular identifier (UMI). In some embodiments, the second probe further comprises a unique molecular identifier (UMI). In some embodiments, the UMI in the first probe and the UMI in the second probe comprise different sequences. In some embodiments, the UMI in the first probe and the UMI in the second probe comprise the same sequence.

In some embodiments of any of the arrays or methods described herein, the first and/or second probe can further include a functional domain (e.g., a sequencing handle). In some embodiments, the first and second probe comprise a functional domain. In some embodiments, the functional domain the first and second probes is the same. In some embodiments, the functional domain in the first probe and the functional domain in the second probe are different.

### Target Nucleic Acids

Described herein are methods for determining a location of a target nucleic acid in a biological sample. Examples of target nucleic acids include DNA (such as genomic DNA, methylated DNA, specific methylated DNA sequences, or fragmented DNA), RNA such as various types of coding and non-coding RNA. Examples of the different types of RNA analytes include messenger RNA (mRNA), ribosomal RNA (rRNA), transfer RNA (tRNA), and microRNA (miRNA).

In some embodiments, the target nucleic acid encodes a T-cell receptor (TCR), found on the surface of T lymphocytes (e.g., T cells). T-lymphocytes play a role in a body's immune response. TCR is a transmembrane heterodimer consisting of an alpha and beta chain or a delta and gamma chain linked by a disulfide bond. Within these chains are complementary determining regions (CDRs) which determine the antigen to which the TCR will bind. TCRs activate the T cells in which they are expressed leading to different immune responses.

TCR development occurs through a lymphocyte specific process of gene recombination, which assembles a final sequence from a large number of potential segments. This genetic recombination of TCR gene segments in somatic T cells occurs during the early stages of development in the thymus. The TCRα gene locus contains variable (V) and joining (J) gene segments (Vβ and Jβ), whereas the TCRβ locus contains a diversity (D) gene segment in addition to Vα and Jα segments. Accordingly, the α chain is generated from VJ recombination and the β chain is involved in VDJ recombination. This is similar for the development of γδ TCRs, in which the TCRγ chain is generated from VJ recombination and the TCRδ gene is generated from VDJ recombination.

The TCRα chain gene locus consists of 46 variable segments, 8 joining segments. and the constant region. The TCR β chain gene locus consists of 48 variable segments followed by two diversity segments, 12 joining segments, and two constant regions. The D and J segments are located within a relatively short 50 kb region while the variable genes are spread over a large region of 1.5 mega bases (TCRα) or 0.67 megabases (TCRβ). The methods described herein allow for the spatial identification and analysis of rearranged V(D)J TCR sequences from both the 3′ end and the 5′ end.

In some embodiments, the target nucleic acid encodes a full-length rearranged V(D)J TCR. In some embodiments, the target nucleic acid encodes a part of a rearranged V(D)J TCR. In some embodiments, the target nucleic acid encodes one or more (e.g., one, two, or three) full-length complementarity determining region(s) (CDR(s)) of the TCR. In some embodiments, the target nucleic acid encodes a part of a complementarity determining region (CDR) of the TCR.

In some embodiments, the target nucleic acid encodes an engineered TCR, for example, a recombinant TCR used in immunotherapy (e.g., an adoptive cell therapy). In some embodiments, the TCR may be αβ heterodimers or may be a single-chain TCR. Single chain TCRs include αβ TCR polypeptides of the Vα-L-V β, Vβ-L-Vα, Vα-Cα-L-V β, or Vα-L-V β-C β types, where Vα and Vβ are TCR α and β variable regions respectively, Cα and Cβ are TCR α and β constant regions respectively, and L is a linker sequence. In some embodiments, the TCR does not have transmembrane or cytoplasmic domains.

In some embodiments, the target nucleic acid encodes a chimeric antibody receptor (CAR).

In some embodiments, the target nucleic of interest encodes a B-cell receptor (BCR), found on the surface of B lymphocytes (e.g., B cells). B lympochytes also play a role in a body's immune response. A BCR is a transmembrane protein composed of an immunoglobulin molecule and a signal transduction moiety, or a heterodimer CD79. In some embodiments, the target nucleic acid encodes a heavy chain or light chain of an immunoglobulin (e.g., IgM, IgG, IgA, IgD, and IgE). In some embodiments, the target nucleic acid encodes an immunoglobulin heavy chain. In some embodiments, the target nucleic acid encodes an immunoglobulin light chain. In some embodiments, the target nucleic acid encodes one or more (e.g., one, two, or three) full-length complementarity determining region(s) (CDR(s)) of an immunoglobulin. In some embodiments, the target nucleic acid encodes a part of a complementarity determining region (CDR) of the immunoglobulin.

In some embodiments, the immunoglobulin can be an engineered immunoglobulin. In some embodiments, the immunoglobulin is a fragment of a full-length immunoglobulin, e.g., a single-chain variable fragment. In some embodiments, the immunoglobulin is a single domain antibody (sdAb).

The size of the target nucleic acid can be any suitable size of a nucleic acid molecule in a biological sample. In some embodiments, the size of the target nucleic acid is about 50 nucleotides to about 100,000 nucleotides (e.g., about 50 nucleotides to about 50,000 nucleotides, about 200 nucleotides to about 10,000 nucleotides, about 500 nucleotides to about 8,000 nucleotides, about 500 nucleotides to about 2,000 nucleotides, about 500 nucleotides to about 1,000 nucleotides, about 1,000 nucleotides to about 8,000 nucleotides, about 1,000 nucleotides to about 4,000 nucleotides, about 1,000 nucleotides to about 2,000 nucleotides, about 2,000 nucleotides to about 4,000 nucleotides, about 4,000 nucleotides to about 6,000 nucleotides, about 6,000 nucleotides to about 8,000 nucleotides, about 8,000 nucleotides to about 10,000 nucleotides).

### IV. Capture and Analysis of 5′ Polynucleotide Sequences

Methods are provided for detecting, determining location of, and/or sequencing 5′ sequences of polynucleotides. The methods include capturing a polynucleotide with a first probe (e.g., binding, ligating, or hybridizing a polynucleotide to a probe). The first probe contains a first capture domain to which a polynucleotide of interest (e.g., a target polynucleotide sequence of a biological analyte, e.g., within a biological sample) binds. In one embodiment, the target polynucleotide binds to (e.g., hybridizes with) a complementary or partially complementary sequence of the first capture domain. In some embodiments, the first probe contains one or more barcode sequences (e.g., a spatial barcode or a cell barcode sequence), e.g., 5′ to the capture domain. The first probe may contain an extendible 3′ end, e.g., at the 3′ end of the capture domain. In one embodiment, the 5′ end of the first capture probe is attached to a substrate.

In some embodiments, a target polynucleotide binds or attaches to the first capture domain of the first probe. In one embodiment, the target polynucleotide contains a sequence that is complementary or partially complementary to a sequence of the first probe, and the target polynucleotide hybridizes to the complementary or partially complementary sequence of the first probe.

The 3′ end of the first probe may be extended to generate a first extension product (e.g., a cDNA sequence) that includes a nucleotide sequence that is complementary to the target polynucleotide sequence or a portion thereof. The first extension product can bind to a second probe or to one or more nucleotide(s) attached to a substrate.

In some embodiments, an adapter is attached to the 3′ end of the first extension product. The adapter or a 3′ sequence thereof may be complementary to a sequence (e.g., a second capture domain) of a second probe. In one embodiment, the adapter is ligated to the 3′ end of the first extension product. In another embodiment, a template switching oligonucleotide (TSO) binds (e.g., hybridizes) to untemplated nucleotides that are added to the 3′ end of the first extension product. The 3′ end of the TSO is then extended to generate an adapter that is complementary to the TSO. The untemplated nucleotides may be A, T, C, and/or G nucleotides, or any sequence thereof. In some embodiments, the untemplated nucleotides are a poly(C) or poly(G) sequence. In one embodiment, the untemplated nucleotides are a poly(C) sequence and the TSO includes a 3′ poly(G) sequence. In another embodiment, the untemplated nucleotides are a poly(G) sequence and the TSO includes a 3′ poly(C) sequence.

In some embodiments, the first extension (e.g., cDNA) product, generated from extension of the first probe bound to the target polynucleotide sequence, binds to a second capture domain of a second probe. In some embodiments, the second capture domain may include: a 3′ sequence that is complementary to a 3′ adapter, or a portion thereof, of the first extension product. The adapter binds (e.g., hybridizes) to the 3′ complementary sequence, and then a3′ end of the second capture domain is extended to produce a second extension product. The second extension product includes a sequence that is complementary to the 5′ sequence of the first extension product, e.g., includes a 5′ sequence of the target polynucleotide, proximal to the sequence of the second capture domain. In one embodiment, the 5′ end of the second probe is attached to a substrate. In some embodiments, the second probe may be cleaved, e.g., at a site 5′ to the second capture domain, thereby releasing the second probe from the substrate, and the released second probe may be used, for example, for amplification and/or sequencing.

In other embodiments, the first extension (e.g., cDNA) product, generated from extension of the first probe bound to the target polynucleotide sequence, is ligated to a second probe. For example, the 3′ end of the first extension product may be ligated to a 5′ end of the second probe. In some embodiments, a splint oligonucleotide binds (e.g., hybridizes) to a 5′ sequence of the second probe and to a 3′ sequence of the first extension product. The 5′ end of the second probe is ligated to the 3′ end of the first extension product, and the 3′ end of the splint oligonucleotide is extended to produce a second extension product. The second extension product includes a sequence that is complementary to the 5′ sequence of the first extension product, e.g., includes a 5′ sequence of the target polynucleotide, proximal to the sequence of the second capture domain. In one embodiment, the 3′ end of the second probe is attached to a substrate.

The method may further include detection of the target polynucleotide, for example, by binding a detectable label, such as a fluorescent label, to the second extension product.

In the methods described herein, the first and/or second extension product may be used for preparation of a 3′ and/or 5′ sequencing library, respectively.

The method may further include determining the sequence of all or a portion of the target polynucleotide sequence, such as the sequence of at least a 5′ portion of the target polynucleotide sequence. In some embodiments, prior to sequencing, an amplification product may be generated from the second extension product, wherein the amplification product includes a 5′ sequence of the target polynucleotide or a complement thereof, and the amplification product may be used for sequencing.

The first and/or second probe may contain a barcode sequence (e.g., a spatial barcode or a cell barcode sequence). In some embodiments, the first and second probes contain barcode sequences, which may be the same or different. In some embodiments, the first and/or second probe contains a unique molecular identifier (UMI). In some embodiments, the UMI in the first probe and the UMI in the second probe comprise different sequences.

In other embodiments, a nucleotide or a sequence of nucleotides is bound at the 3′ end to a substrate, and the 3′ end of the first extension product is bound to a 5′ end of the nucleotide(s), for example via hybridization or ligation. In some embodiments, a splint oligonucleotide binds (e.g., hybridizes) to a 5′ end of the nucleotide(s) and to a 3′ sequence of the first extension product. The 5′ end of the nucleotide(s) is ligated to the 3′ end of the first extension product, and the 3′ end of the splint oligonucleotide is extended to produce a second extension product. The second extension product includes a sequence that is complementary to the 5′ sequence of the first extension product, e.g., includes a 5′ sequence of the target polynucleotide, proximal to the sequence of the second capture domain.

The first and/or second extension product may be produced using a polymerase, e.g., a DNA polymerase (RNA-dependent DNA polymerase (e.g., reverse transcriptase) or a DNA-dependent DNA polymerase), or an RNA polymerase.

The target polynucleotide may comprise or consist of DNA or RNA, and/or may contain non-natural nucleotides. The first capture domain may include a poly(dT) sequence, a random sequence, or a sequence that is complementary or partially complementary to a target sequence of interest. In certain embodiments, the target polynucleotide is mRNA. The mRNA may encode, for example, a TCR or a B-cell receptor (BCR), and in some embodiments, the method may include determining a sequence that encodes one or more complementarity determining region(s) (CDR(s)) of the TCR or BCR. The mRNA may encode, for example, an immunoglobulin heavy chain or light chain, and the method may include determining a sequence that encodes one or more CDR(s) of the immunoglobulin heavy chain or light chain.

In some embodiments, the target nucleic acid encodes a full-length rearranged V(D)J TCR. In some embodiments, the target nucleic acid encodes a portion of a rearranged V(D)J TCR. In some embodiments, the target nucleic acid encodes one or more (e.g., one, two, or three) full-length complementarity determining region(s) (CDR(s)) of the TCR. In some embodiments, the target nucleic acid encodes a part of a complementarity determining region (CDR) of the TCR.

In some embodiments, the target nucleic acid encodes an engineered TCR, for example, a recombinant TCR used in immunotherapy (e.g., an adoptive cell therapy). In some embodiments, the TCR may be αβ heterodimers or may be a single-chain TCR. Single chain TCRs include αβ TCR polypeptides of the Vα-L-V β, Vβ-L-Vα, Vα-Cα-L-V β, or Vα-L-V β-C β types, where Vα and Vβ are TCR α and β variable regions respectively, Cα and Cβ are TCR α and β constant regions respectively, and L is a linker sequence. In some embodiments, the TCR does not have transmembrane or cytoplasmic domains.

In some embodiments, the target nucleic acid encodes a chimeric antibody receptor (CAR).

In some embodiments, the target nucleic acid encodes a heavy chain or light chain of an immunoglobulin (e.g., IgM, IgG, IgA, IgD, and IgE). In some embodiments, the target nucleic acid encodes an immunoglobulin heavy chain. In some embodiments, the target nucleic acid encodes an immunoglobulin light chain. In some embodiments, the target nucleic acid encodes one or more (e.g., one, two, or three) full-length complementarity determining region(s) (CDR(s)) of an immunoglobulin. In some embodiments, the target nucleic acid encodes a part of a complementarity determining region (CDR) of the immunoglobulin.

In some embodiments, the immunoglobulin can be an engineered immunoglobulin. In some embodiments, the immunoglobulin is a fragment of a full-length immunoglobulin, e.g., a single-chain variable fragment. In some embodiments, the immunoglobulin is a single domain antibody (sdAb).

In some embodiments, the target polynucleotide includes a genetic variation, such as an isoform, a splice variant, or a single nucleotide polymorphism (SNP), e.g., in a 5′ region of the mRNA, and the method includes detection and/or sequencing of the genetic variation. For example, the first capture domain may include a sequence that is specific for the genetic variation, such as a sequence that binds to a change in a nucleic acid or protein (e.g., a mutation or SNP.

The biological sample from which the target polynucleotide is derived may be, for example, a tissue section, a primary cell, a cell line, or an organoid. In some embodiments, the method includes permeabilizing the biological sample, to release a biological analyte, prior to contacting the first probe with the target polynucleotide sequence of the biological analyte. In some embodiments, the biological sample is a tissue section, such as a fixed (e.g., formalin-fixed and paraffin-embedded (FFPE) or paraformaldehyde (PFA)) tissue section, or a fresh frozen tissue section.

In some embodiments, the first and second probes are attached to a substrate (a support), such as a substrate that comprises an array. The array may be, for example, a bead array or a slide. For example, the first and second probes may be attached to a feature in an array. The ratio of first probes to second probes may be about 1:1000 to about 1000:1, or about 1:1 to about 1:100. The first and second probes may be attached directly or indirectly to the substrate. In some embodiments, the first and second probes are attached indirectly to the substrate, such as attached to beads (for example, gel beads) that are attached to the substrate. In some embodiments, the first and second probes are attached indirectly to the substrate via a linker (for example, a cleavable linker such as photocleavable linker (e.g., bromodeoxyuridine (BrdU), an enzymatic linker (e.g., uracil specific excision enzyme), or a chemical linker (e.g., a sulfhydryl, amide or carboxyl group), that are attached to the substrate.

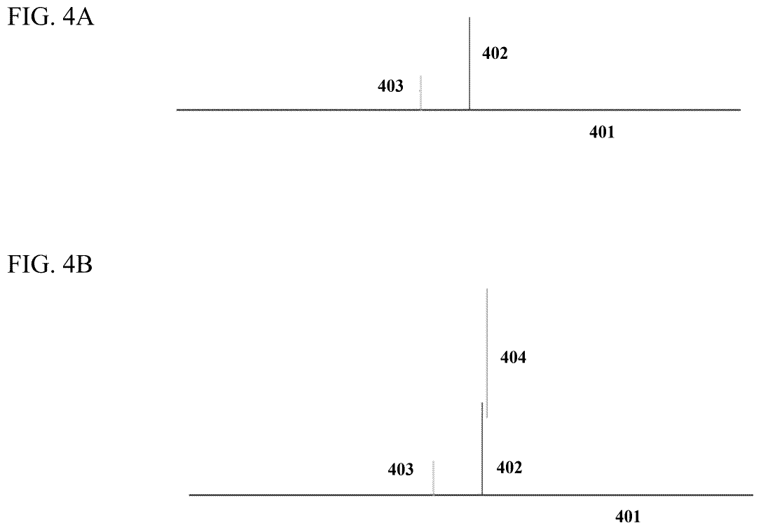

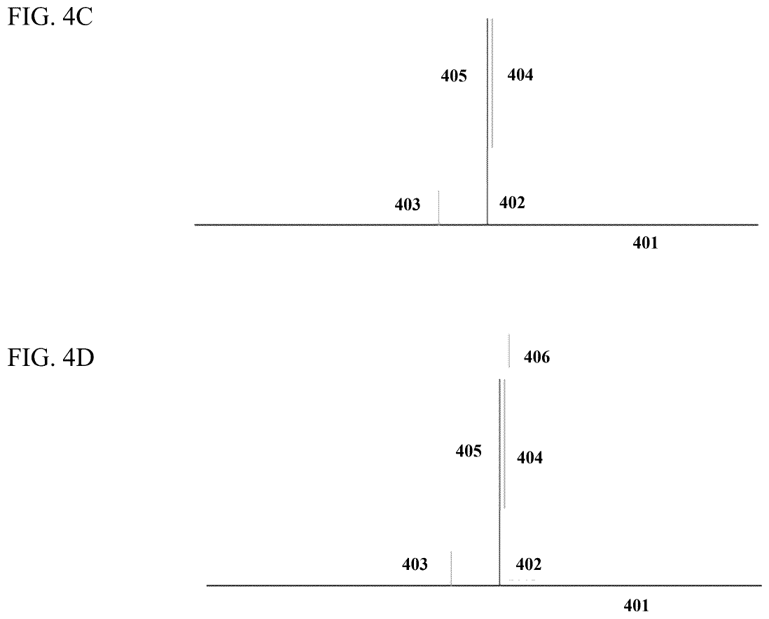

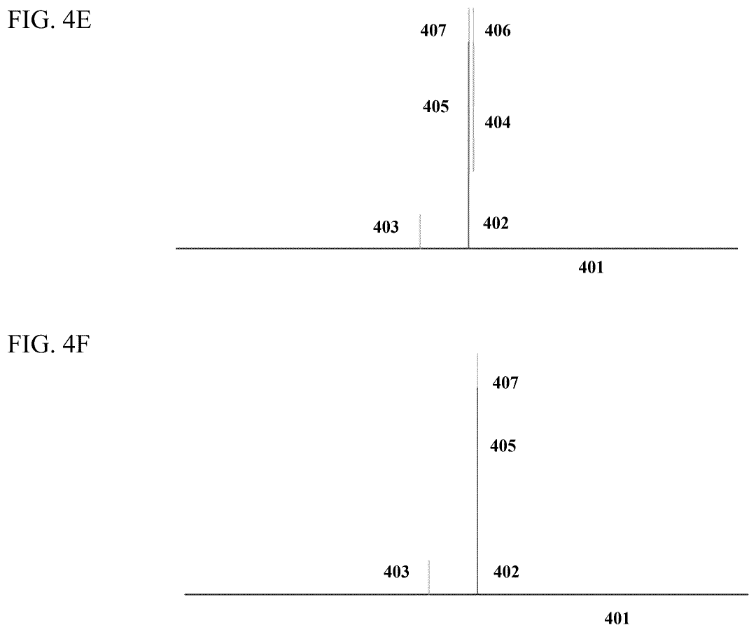

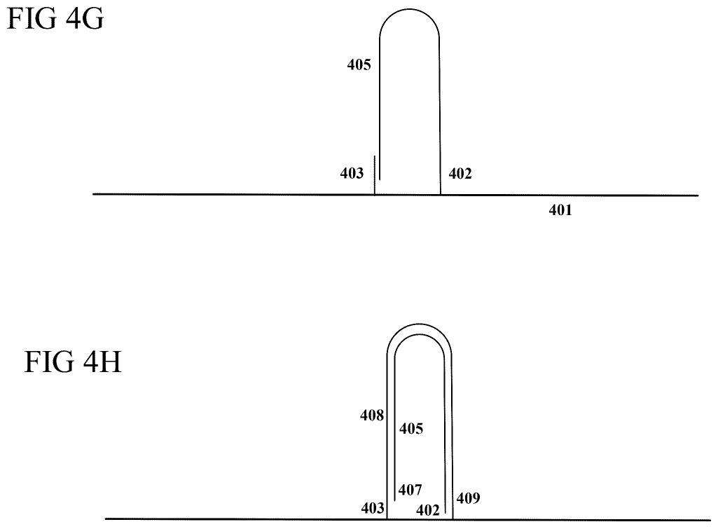

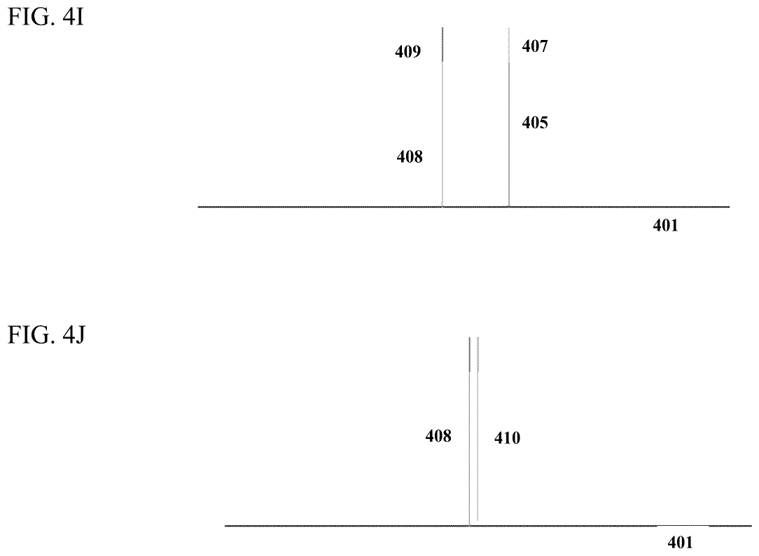

An exemplary, non-limiting workflow is depicted in FIGS. 4A-4J. In the workflow depicted in FIGS. 4A-4J, presence and/or location (e.g., spatial location) of a biological analyte may be determined and/or a 5′ sequence (e.g., sequence of a 5′ region) of a polynucleotide sequence of a biological analyte may be detected and/or determined. A “5′ region” refers to a sequence that is at or near the 5′ end of a polynucleotide sequence, or a sequence that is closer in proximity to the 5′ end than the 3′ end of a polynucleotide sequence. A biological sample is contacted with a substrate 401. The substrate includes an attached first probe 402 and an attached second probe 403. (FIG. 4A) In one embodiment, the first probe 402 includes, in a 5′-3′ direction: a barcode (e.g., spatial barcode); a first capture domain; and a 3′ end. In the embodiment depicted in FIGS. 4A-4J, the second probe 403 includes a second capture domain and a 3′ end, and both the first probe 402 and the second probe 403 are attached at their 5′ ends to the substrate 401.

A biological sample is contacted with the substrate 401 and a target polynucleotide sequence 404 of a biological analyte binds (e.g., hybridizes) to the first capture domain of the first probe 402. (FIG. 4B) In some instances, the first capture domain includes a sequence specific for an RNA molecule. In some instances, the first capture domain includes a poly-T sequence. In some instances, the first capture domain includes a sequence complementary to a region of an immune molecule, such as the constant region of a TCR or BCR sequence (as described herein). In some instances, the first capture domain includes a sequence complementary to a region of an immunoglobulin molecule, such as one or more CDRs of an immunoglobulin heavy or light chain.

The 3′ end of the first probe 402 is extended to produce a first extension product 405. (FIG. 4C) An adapter is attached to the 3′ end of the first extension product 405. In one embodiment, depicted in FIG. 4D, untemplated nucleotides are added to the 3′ end of the first extension product 405. A template switching oligonucleotide (TSO) 406 binds (e.g., hybridizes) to the untemplated nucleotides, and then the 3′ end of the first extension product is extended, producing a polynucleotide sequence 407 that is complementary to the TSO sequence. (FIG. 4E). The target polynucleotide sequence 404 and TSO 406 are stripped away (e.g., denatured). (FIG. 4F)

In the embodiment depicted in FIG. 4G, the second capture domain of the second probe 403 includes a sequence that is complementary to the adapter 407, i.e., the second capture domain contains the TSO sequence or a partial sequence thereof. The adapter 407 at the 3′ end of the first extension product 405 binds to the second capture domain at the 3′ end of the second probe 403. The 3′ end of the second probe 403 is extended, producing a second extension product 408, which includes a 3′ sequence that is complementary to the sequence of the first probe or a portion thereof 409. (FIG. 4H) The first extension product 405 with 3′ adapter 407 includes a 3′ sequence complementary to the target polynucleotide proximal to the first capture domain sequence, and may be used for preparation of a 3′ sequence library; and/or the second extension product 408 with 3′ sequence complementary to the first probe 407 includes a 5′ sequence of the target polynucleotide proximal to the second capture domain, and may be used for preparation of a 5′ sequence library. (FIG. 4I) In one embodiment, depicted in FIG. 4J, a copy 410 of the second extension product (e.g., amplification product) 408 is produced. The first and/or second extension product, and/or copy (e.g., amplification product thereof) may be detected and/or sequenced, and the resulting information obtained may be used to determine presence and/or location (e.g., spatial location) of the biological analyte in the biological sample.

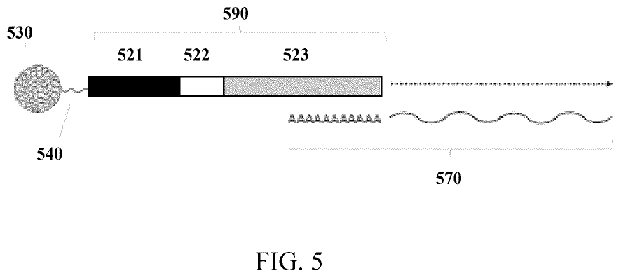

In some embodiments of the method, analysis of one or more analyte(s) from single cells is performed according to an exemplary, nonlimiting workflow as depicted in FIG. 5. Single cells and supports 530 (e.g., a bead, such as a gel bead) including a nucleic acid barcode molecule 590 are co-partitioned into a partition amongst a plurality of partitions (e.g., a droplet of a droplet emulsion or a well of a micro/nanowell array). In some instances, the partition includes at most a single cell and a single support 530. In some embodiments, nucleic acid barcode molecule 590 is attached to support 530 via a releasable linkage 540 (e.g., including a labile bond). Upon release of nucleic acid barcode molecule 590 from the support 530, barcoded molecules may be generated within the partition. In some embodiments, nucleic acid barcode molecule 590 includes a sequence 523 complementary to a sequence of RNA molecule 560 from a cell. In some instances, sequence 523 includes a sequence specific for an RNA molecule. In some instances, sequence 523 includes a poly-T sequence. In some instances, sequence 523 includes a sequence complementary to a region of an immune molecule, such as the constant region of a TCR or BCR sequence (as described herein). In some instances, sequence 523 includes a sequence complementary to a region of an immunoglobulin molecule, such as one or more CDRs of an immunoglobulin heavy or light chain. Sequence 523 is bound (e.g., hybridized) to RNA molecule 560 and a cDNA molecule 570 is generated in a reverse transcription reaction, generating a barcoded nucleic acid molecule including cell (e.g., partition specific) barcode sequence 522 (or a reverse complement thereof) and a sequence of cDNA 570 (or a portion thereof). Barcoded nucleic acid molecules can then be optionally processed as described elsewhere herein, e.g., to amplify the molecules and/or append sequencing platform specific sequences to the fragments. See, e.g., U.S. Pat. Pub. Nos. 2018/0105808 and 2019/0367969 and U.S. Pat. Nos. 10,273,541, 10,480,029, and 10,550,429, each of which is hereby incorporated by reference in its entirety. Barcoded nucleic acid molecules, or derivatives generated therefrom, can then be sequenced on a suitable sequencing platform.

### V. Methods for Removal of a Polynucleotide Region From a Template Polynucleotide

Methods are provided for detecting a biological analyte in a biological sample, wherein sequences that are not of interest are removed from a template polynucleotide to facilitate analysis. The methods include binding (e.g., hybridizing) first and second oligonucleotides to a template polynucleotide. The template polynucleotide includes a polynucleotide sequence of a biological analyte or the complement thereof. In some embodiments, the template polynucleotide includes a barcode sequence 5′ to the polynucleotide sequence of the biological analyte, (e.g., a spatial barcode sequence or a cell barcode sequence). The first oligonucleotide includes a sequence that is complementary to a first sequence of the template polynucleotide, and a 3′ end. The second oligonucleotides is a bridging oligonucleotide that includes sequences that are complementary to second and third sequences of the template polynucleotide, wherein the second and third sequences are flanking a polynucleotide region of the template polynucleotide to be removed. In some embodiments, the second oligonucleotide includes a 5′ phosphate group. The first sequence of the template polynucleotide is 3′ to the second sequence of the template polynucleotide. The second and third sequences of the template polynucleotide are 3′ and 5′, respectively, to a fourth sequence of the template polynucleotide which is a polynucleotide region to be removed.

The 3′ end of at least the first oligonucleotide is extended. In some embodiments, 3′ ends of both the first and second oligonucleotides are extended. In one embodiment, a non-strand displacing, non 5′-3′ exonuclease DNA polymerase is used for oligonucleotide extension. For example, T4 and T7 DNA polymerases (e.g., NEB catalog numbers 0203 and 0274) lack strand displacement activity and can be used. The first oligonucleotide is extended to the 5′ end of the second oligonucleotide, and then joined to the second oligonucleotide. In some embodiments, the 3′ end of the first oligonucleotide is ligated to a phosphate group at the 5′ end of the second oligonucleotide. The resulting polynucleotide product includes the complement of 5′ and 3′ portions of the template polynucleotide sequence, and does not include the complement of the fourth sequence of the template polynucleotide.

In some embodiments, prior to binding the first and second oligonucleotides to the template polynucleotide, the template polynucleotide is captured from a biological sample. For example, a biological sample may be contacted with a probe. The probe may include, in a 5′ to 3′ direction, a barcode sequence (e.g., a spatial barcode sequence or a cell barcode sequence), a capture domain. In some embodiments, the probe includes a unique molecular identifier (UMI).

The biological sample is contacted with the probe where a target polynucleotide sequence of a biological analyte (a template polynucleotide) binds (e.g., hybridizes) to the capture domain. A 3′ end of the capture domain is extended, generating an extension product that includes a sequence that is complementary to the template polynucleotide, or a portion thereof (e.g., a cDNA extension product). The first and second oligonucleotides then bind to the extension product, are extended, as described above, and the 3′ end of the extended first oligonucleotide is joined to the 5′ end of the extended second oligonucleotide. The first oligonucleotide is complementary to a first sequence of the extension product, and the second oligonucleotide is complementary to second and third sequences of the extension product. The first sequence of the extension product is 3′ to the second sequence of the extension product, and the second and third sequences are 3′ and 5′, respectively, to a fourth sequence of the extension product, which is to be removed. The polynucleotide product does not include the complement of the fourth sequence of the extension product (i.e., the sequence of the template polynucleotide to which the fourth sequence of the extension product is complementary). In one embodiment, uridine residues are incorporated into the extension product, and the method includes digestion of the extension product with a uracil specific excision reagent (USER), after oligonucleotide extension and joining of the extended first oligonucleotide to the second oligonucleotide.

In some embodiments, the polynucleotide region to be removed (e.g., the fourth sequence of a template polynucleotide or the complement thereof) is about 10 nucleotides to about 1000 nucleotides, or longer. For example, the fourth sequence may be any of about 10 nucleotides to about 100 nucleotides about 75 nucleotides to about 125 nucleotides, about 125 nucleotides to about 175 nucleotides, about 175 nucleotides to about 225 nucleotides, about 225 nucleotides to about 275 nucleotides, about 275 nucleotides to about 325 nucleotides, about 325 nucleotides to about 375 nucleotides, about 375 nucleotides to about 425 nucleotides, about 425 nucleotides to about 475 nucleotides, about 475 nucleotides to about 525 nucleotides, about 525 nucleotides to about 575 nucleotides, about 575 nucleotides to about 625 nucleotides, about 625 nucleotides to about 675 nucleotides, about 675 nucleotides to about 725 nucleotides, about 725 nucleotides to about 775 nucleotides, about 775 nucleotides to about 825 nucleotides, about 825 nucleotides to about 875 nucleotides, about 875 nucleotides to about 925 nucleotides, about 925 nucleotides to about 975 nucleotides, about 950 nucleotides to about 1000 nucleotides, about 20 nucleotides to about 200 nucleotides, about 50 nucleotides to about 150 nucleotides, about 100 nucleotides to about 200, nucleotides about 150 nucleotides to about 250 nucleotides, about 200 nucleotides to about 300 nucleotides, about 250 nucleotides to about 350 nucleotides, about 300 nucleotides to about 400 nucleotides, about 350 nucleotides to about 450 nucleotides, about 400 nucleotides to about 500 nucleotides, about 450 nucleotides to about 550 nucleotides, about 500 nucleotides to about 600 nucleotides, about 550 nucleotides to about 650 nucleotides, about 600 nucleotides to about 700 nucleotides, about 650 nucleotides to about 750 nucleotides, about 700 nucleotides to about 800 nucleotides, about 750 nucleotides to about 850 nucleotides, about 800 nucleotides to about 900 nucleotides, about 850 nucleotides to about 950 nucleotides, about 900 nucleotides to about 1000 nucleotides, about 20 nucleotides to about 200 nucleotides, about 100 nucleotides to about 300 nucleotides, about 200 nucleotides to about 400 nucleotides, about 300 nucleotides to about 500 nucleotides, about 400 nucleotides to about 600, nucleotides about 500 nucleotides to about 700 nucleotides, about 600 nucleotides to about 800 nucleotides, about 700 nucleotides to about 900 nucleotides, about 800 nucleotides to about 1000 nucleotides, about 20 nucleotides to about 250 nucleotides, about 100 nucleotides to about 400 nucleotides, about 200 nucleotides to about 500 nucleotides, about 300 nucleotides to about 600 nucleotides, about 400 nucleotides to about 700 nucleotides, about 500 nucleotides to about 800 nucleotides, about 600 nucleotides to about 900 nucleotides, about 700 nucleotides to about 1000 nucleotides, about 20 nucleotides to about 300 nucleotides, about 150 nucleotides to about 500 nucleotides, about 250 nucleotides to about 600 nucleotides, about 400 nucleotides to about 800 nucleotides, about 600 nucleotides to about 1000 nucleotides, about 20 nucleotides to about 500 nucleotides, about 250 nucleotides to about 750 nucleotides, or about 500 nucleotides to about 1000 nucleotides, about 1000 nucleotides to about 1500 nucleotides, or about 1500 nucleotides to about 2000 nucleotides, or longer.

In some embodiments, the first polynucleotide sequence to which the first oligonucleotide binds is at the 3′ end of the template polynucleotide or to the 3′ end of its complement, such as the 3′ end of a complementary extension product thereof. In certain embodiments, the first oligonucleotide binds to a 3′ adapter. In one embodiment, the adapter is ligated to the 3′ end of the template polynucleotide or to the 3′ end of its complement, such as the 3′ end of a complementary extension product thereof. In another embodiment, a template switching oligonucleotide (TSO) binds (e.g., hybridizes) to untemplated nucleotides that are added to the 3′ end of the template polynucleotide or to the 3′ end of its complement, such as the 3′ end of a complementary extension product thereof. The 3′ end of the TSO is then extended to generate an adapter that is complementary to the TSO. The untemplated nucleotides may be A, T, C, and/or G nucleotides, or any sequence thereof. In some embodiments, the untemplated nucleotides are a poly(C) or poly(G) sequence. In one embodiment, the untemplated nucleotides are a poly(C) sequence and the TSO includes a 3′ poly(G) sequence. In another embodiment, the untemplated nucleotides are a poly(G) sequence and the TSO includes a 3′ poly(C) sequence.

In some embodiments, the sequence of the first and/or second oligonucleotide may be about 10 nucleotides to about 50 nucleotides, about 10 nucleotides to about 20 nucleotides, about 15 nucleotides to about 20 nucleotides, about 20 nucleotides to about 30 nucleotides, about 25 nucleotides to about 35 nucleotides about 30 nucleotides to about 40 nucleotides, or about 35 nucleotides to about 45 nucleotides, or about 40 nucleotides to about 50 nucleotides, about 10 nucleotides to about 30 nucleotides, about 15 nucleotides to about 35 nucleotides, about 20 to about 40 nucleotides, about 25 nucleotides to about 45 nucleotides, about 30 nucleotides to about 50 nucleotides in length, or any of about 10, 15, 20, 25, 30, 35, 40, 45, or 50 nucleotides in length.

In some embodiments, the second and/or third sequence to which a 5′ or 3′ sequence of the second oligonucleotide, respectively, binds may be about 10 nucleotides to about 50 nucleotides, about 10 nucleotides to about 20 nucleotides, about 15 nucleotides to about 20 nucleotides, about 20 nucleotides to about 30 nucleotides, about 25 nucleotides to about 35 nucleotides about 30 nucleotides to about 40 nucleotides, or about 35 nucleotides to about 45 nucleotides, or about 40 nucleotides to about 50 nucleotides, about 10 nucleotides to about 30 nucleotides, about 15 nucleotides to about 35 nucleotides, about 20 to about 40 nucleotides, about 25 nucleotides to about 45 nucleotides, about 30 nucleotides to about 50 nucleotides in length, or any of about 10, 15, 20, 25, 30, 35, 40, 45, or 50 nucleotides in length.

In some embodiments, the second oligonucleotide includes a linker, e.g., between the sequences that are complementary to the second and third sequences of the template polynucleotide or a complement thereof, such as a complementary extension product thereof. The linker may be a sequence of nucleotides that is not complementary to a sequence of the template polynucleotide or a complement thereof, such as a complementary extension product thereof. In a nonlimiting embodiment, the linker includes the sequence (AT)ₙ. In some embodiments, the linker is about 1 nucleotide to about 50 nucleotides, about 1 nucleotide to about 5 nucleotides, about 1 nucleotide to about 10 nucleotides, about 5 nucleotides to about 10 nucleotides, about 10 nucleotides to about 20 nucleotides, about 15 nucleotides to about 20 nucleotides, about 20 nucleotides to about 30 nucleotides, about 25 nucleotides to about 35 nucleotides about 30 nucleotides to about 40 nucleotides, or about 35 nucleotides to about 45 nucleotides, or about 40 nucleotides to about 50 nucleotides, about 10 nucleotides to about 30 nucleotides, about 15 nucleotides to about 35 nucleotides, about 20 to about 40 nucleotides, about 25 nucleotides to about 45 nucleotides, about 30 nucleotides to about 50 nucleotides in length, or any of about 1, 5, 10, 15, 20, 25, 30, 35, 40, 45, or 50 nucleotides in length.

The polynucleotide product after oligonucleotide extension may be used for amplification and/or sequencing, for example, to detect presence and/or quantity of a template polynucleotide, and/or determine a 5′ and/or 3′ sequence of the template polynucleotide or a complement thereof, and/or to determine location (e.g., spatial location) of the biological analyte or a polynucleotide sequence thereof.

In some embodiments of the method, the template polynucleotide may be immobilized on a substrate (support or surface). For example, the template polynucleotide may be bound (e.g., hybridized) to a capture domain of a probe. In some embodiments, the probe may be configured as a component of an array of probes on the support. The array may be, for example, a bead array or a slide. For example, the probe may be attached to a feature in an array. The probe may be attached directly or indirectly to the substrate. In some embodiments, the probe is attached indirectly to the substrate, such as attached to beads (for example, gel beads) that are attached to the substrate. In some embodiments, the probe is attached indirectly to the substrate via a linker (for example, a photocleavable, chemically cleavable, or enzymatically cleavable linker) that are attached to the substrate.

In some instances, the first capture domain includes a poly-T sequence. In some instances, the capture domain includes a sequence complementary to a region of an immune molecule, such as the constant region of a TCR or BCR sequence (as described herein). In some instances, the capture domain includes a sequence complementary to a region of an immunoglobulin molecule, such as one or more CDRs of an immunoglobulin heavy or light chain. In some embodiments, the template polynucleotide includes a genetic variation, such as an isoform, a splice variant, or a single nucleotide polymorphism (SNP), e.g., in a 5′ region of the mRNA, and the method includes detection and/or sequencing of the genetic variation. For example, the capture domain may include a sequence that is specific for the genetic variation, such as a sequence that binds to a change in a nucleic acid or protein (e.g., a mutation or SNP).

The biological sample from which the template polynucleotide is derived may be, for example, a tissue section, a primary cell, a cell line, or an organoid. In some embodiments, the method includes permeabilizing the biological sample, to release a biological analyte, prior to binding of the first and second oligonucleotides to the template polynucleotide of the biological analyte, for example, prior to contacting a capture probe with the template polynucleotide of the biological analyte. In some embodiments, the biological sample is a tissue section, such as a fixed (e.g., formalin-fixed and paraffin-embedded (FFPE) or paraformaldehyde (PFA)) tissue section, or a fresh frozen tissue section.

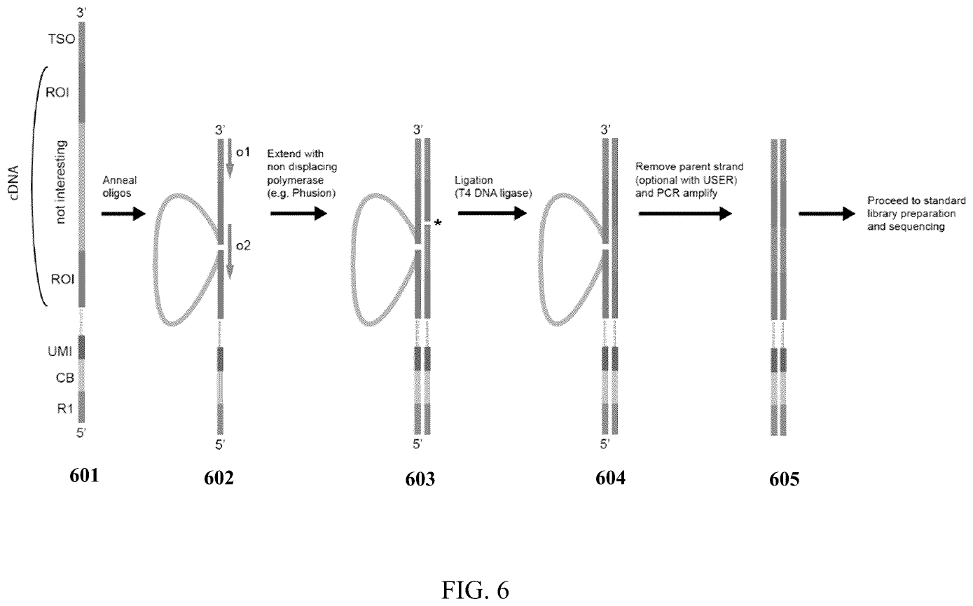

An exemplary, non-limiting workflow is depicted in FIG. 6. A cDNA complement 601 of a template polynucleotide includes two regions of interest (ROI) flanking a region that is not of interest, and an adapter at the 3′ end (TSO). First and second oligonucleotides (o1 and o2,respectively) are annealed to a first cDNA sequence and to second and third cDNA sequences, respectively, bridging and bringing the two ROIs into proximity (602). First and second oligonucleotides are extended with a polymerase. (603) Extension of the first oligonucleotide ceases when a 5′ phosphate (*) of the second oligonucleotide is reached. The 3′ end of the extended first oligonucleotide is ligated to the 5′ end of the second oligonucleotide, resulting in a polynucleotide product that contains the sequences of the two ROIs and that does not contain the sequence of the region that is not of interest. (604) The polynucleotide product may be amplified (605), and optionally sequenced. In some embodiments, the cDNA 601 may be an extension product from a template polynucleotide bound to a capture domain of a capture probe, as described herein. In other embodiments, the template polynucleotide may be an analyte from a single cell, and may be converted to a cDNA molecule as described above (for example, but not limited to the workflow depicted in FIG. 5).

In some embodiments, the disclosure provides for kits for capturing and determining the location and abundance of analytes from a biological sample as described herein. A kit would include, for example, an array comprising a first and second oligonucleotide probe reversibly affixed to the array, enzymes for practicing the methods such as a ligase as described herein, a terminal transferase as described herein, a polymerase as described herein, cleavage enzymes as described herein, library preparatory reagents as described herein, and buffers and dNTPs for practicing the different steps in the methods for capturing and determining the location of a nucleic acid of interest from the biological sample. Further, a kit can include instructions that would provide a user with details on how to practice one or more of the methods for capturing and determining the location of a nucleic acid of interest from a biological sample.

1. (canceled)

2. A method for spatial analysis of a biological sample, the method comprising:
(a) contacting the biological sample with a substrate comprising a plurality of features, wherein a feature of the plurality of feature comprises:
(i) a first probe attached to the substrate at a 5′ end of the first probe, wherein the first probe comprises a first capture domain that is a poly(T) sequence; and
(ii) a second probe attached to the substrate at a 5′ end of the second probe, wherein the second probe comprises (A) a second capture domain that is a template switching oligonucleotide sequence or a partial sequence thereof and (B) a spatial barcode;
(b) hybridizing the first probe to an mRNA molecule from the biological sample via the poly(T) sequence;
(c) extending the 3′ end of the first probe to generate an extension product comprising a sequence complementary to the mRNA molecule or a portion thereof, wherein said extending comprises incorporating a plurality of untemplated nucleotides to the 3′ end of the extension product;
(d) hybridizing a template switching oligonucleotide to the untemplated nucleotides;
(e) extending the 3′ end of the extension product to incorporate a sequence complementary to the sequence of the template switching oligonucleotide;
(f) removing the mRNA molecule and the template switching oligonucleotide hybridized to the extension product;
(g) hybridizing the second capture domain of the second probe to the sequence complementary to the template switching oligonucleotide sequence of the extension product; and
(h) generating a spatially barcoded extension product or a complement thereof.

3. The method of claim 2, wherein generating a spatially barcoded extension product or the complement thereof comprises extending the extension product using the second probe as a template.

4. The method of claim 2, wherein generating a spatially barcoded extension product or the complement thereof comprises extending the second probe using the extension product as a template.

5. The method of claim 2, wherein the second probe further comprises a UMI.

6. The method of claim 2, further comprising blocking the 3′ end of the second probe.

7. The method of claim 2, further comprising generating an amplification product of the spatially barcoded extension product or the complement thereof.

8. The method of claim 2, wherein extending the 3′ end of the first probe to generate an extension product in (c) comprises use of a reverse transcriptase.

9. The method of claim 2, wherein the biological sample is a tissue section, optionally a fixed tissue section or a fresh frozen tissue section.

10. The method of claim 2, further comprising permeabilizing the biological sample to release the mRNA molecule from the biological sample.

11. The method of claim 2, wherein the first and/or second probe is attached to the substrate via a cleavable linker.

12. The method of claim 11, wherein the cleavable linker comprises a photocleavable linker, an enzymatic linker, or a chemical linker.

13. The method of claim 11, wherein the extension product or a complement thereof is released from the substrate via the cleavable linker.

14. The method of claim 2, wherein said removing the mRNA molecule and the template switching oligonucleotide comprises denaturing the mRNA molecule and the template switching oligonucleotide.

15. The method of claim 7, further comprising sequencing the spatially barcoded extension product or the complement thereof to provide sequencing data, or sequencing the amplification product of the spatially barcoded extension product or the complement thereof to provide sequencing data.

16. The method of claim 15, further comprising using the sequencing data to determine the presence and/or location of the mRNA molecule in the biological sample.

17. The method of claim 2, wherein the hybridizing in step (g) is performed with the extension product attached to the substrate and the second probe attached to the substrate.

18. The method of claim 2, wherein the first probe and/or second probe further comprises a functional domain, optionally wherein the functional domain comprises a sequencing primer site.

19. The method of claim 2, wherein the substrate comprises a slide.

20. A method for spatial analysis of a biological sample, the method comprising:
(a) contacting the biological sample with a substrate comprising a plurality of features, wherein a feature of the plurality of feature comprises:
(i) a first probe attached to the substrate at a 5′ end of the first probe, wherein the first probe comprises a first capture domain that is a poly(T) sequence; and
(ii) a second probe attached to the substrate at a 5′ end of the second probe, wherein the second probe comprises (A) a second capture domain that is a template switching oligonucleotide sequence or a partial sequence thereof and (B) a spatial barcode;
(b) hybridizing the first probe to an mRNA molecule from the biological sample via the poly(T) sequence;
(c) extending the 3′ end of the first probe to generate an extension product comprising a sequence complementary to the mRNA molecule or a portion thereof, wherein said extending comprises incorporating a plurality of untemplated nucleotides to the 3′ end of the extension product;
(d) hybridizing a template switching oligonucleotide to the untemplated nucleotides;
(e) extending the 3′ end of the extension product to incorporate a sequence complementary to the sequence of the template switching oligonucleotide;
(f) removing the mRNA molecule and the template switching oligonucleotide hybridized to the extension product;
(g) while the extension product is attached to the substrate and the second probe is attached to the substrate, hybridizing the second capture domain of the second probe to the sequence complementary to the template switching oligonucleotide sequence of the extension product;
(h) generating a spatially barcoded extension product or a complement thereof; and
(i) preparing a sequencing library from the spatially barcoded extension product or the complement thereof.

21. The method of claim 20, wherein generating a spatially barcoded extension product or the complement thereof comprises extending the extension product using the second probe as a template.

22. The method of claim 20, wherein generating a spatially barcoded extension product or the complement thereof comprises extending the second probe using the extension product as a template.

23. The method of claim 20, wherein the second probe further comprises a UMI and/or a functional domain, optionally wherein the functional domain comprises a sequencing primer site.

24. The method of claim 20, further comprising generating an amplification product of the spatially barcoded extension product or the complement thereof.

25. The method of claim 24, further comprising sequencing the spatially barcoded extension product or the complement thereof to provide sequencing data, or sequencing the amplification product of the spatially barcoded extension product or the complement thereof to provide sequencing data.

26. The method of claim 25, further comprising using the sequencing data to determine the presence and/or location of the mRNA molecule in the biological sample.

27. The method of claim 20, wherein extending the 3′ end of the first probe to generate an extension product in (c) comprises use of a reverse transcriptase.

28. The method of claim 20, wherein the biological sample is a tissue section, optionally a fixed tissue section or a fresh frozen tissue section.

29. The method of claim 20, further comprising permeabilizing the biological sample to release the mRNA molecule from the biological sample.

30. The method of claim 20, wherein the first and/or second probe is attached to the substrate via a cleavable linker, optionally wherein the cleavable linker comprises a photocleavable linker, an enzymatic linker, or a chemical linker.

31. The method of claim 20, wherein the substrate comprises a slide.
